(19) United States
(12) Patent Application Publication
(10) Pub. No.: US 2015/0184235 A1
(43) Pub. Date: Jul. 2, 2015

(54) FLEXIBLE SENSOR CARRIER AND METHOD

(71) Applicants: Thorsten Reda, Wien (AT); Igor Hollaender, Wien (AT)

(72) Inventors: Thorsten Reda, Wien (AT); Igor Hollaender, Wien (AT)

(73) Assignee: LEXOGEN GMBH, WIEN (AT)

(21) Appl. No.: 14/414,170

(22) PCT Filed: Jul. 10, 2012
(86) PCT No.: PCT/AT2012/000182

§ 371 Date: Jan. 12, 2015

(51) Int. Cl. C12Q 1/68; G01N 27/02

(52) U.S. Cl. CPC C12Q 1/6837; G01N 27/02

(57) ABSTRACT

A sensor carrier contains a flexible film supporting a plurality of functionalized sensor elements each being formed by a functional layer. The functional layers are located on the same surface of the film within a window area, A region of each of the functional layers of the sensor elements is functionalized, and one or more sensor compounds are arranged or located in the respective functionalized regions of the sensor elements.

### FIELD OF INVENTION

The present invention relates to a sensor carrier and pertains to the design, assembly and use of functionalized surfaces. In particular the invention makes use of flexible films which contain immobilized and surface exposed sensor biomolecules for the use in biosensor array applications. Such biosensor arrays allow multiplex hybridization and selective amplification studies in molecular biology. Consequently, the invention facilitates Lab-on-a Chip or Lab-on-a-Foil solutions built on thin flexible films.

### BACKGROUND OF THE INVENTION

### Nucleic Acid Amplification, Surface Confined Reactions and Biosensor Arrays

The term biosensor is used for devices which either detect biomolecules or use biomolecules to detect different organic or inorganic analytes. Biosensors are used in a wide spectrum from simple analytical devices targeting one or very few compounds, which are relevant for example in medical diagnostics, examples are hCG, sugar, ethanol to name a few, to complex combinatorial screening platforms which are important for life sciences research. All of them use specific reactions between the sensor compound and the analytes of interest. Such reaction should be converted into measurable signals. Numerous sensor applications are carried out on complex analyte mixtures, e.g. RNA, DNA or polypeptides. The challenge herein is that such analyses target many different molecules and therefore it is required to build large arrays to detect and distinguish those high numbers. Miniaturized sensor arrays have the advantage of limiting the amount of a precious sample.

Nucleic acid sensor arrays can be built with a number of individual oligonucleic acid probes where in each spatial location just one kind of probe is located. Such oligonucleic acid probes are small linear polymers which consist typically of 10 to 50 nucleic acid monomers. The probes should be covalently bound when they engage in stringent washing steps. Hybridization assays can be carried out to detect the annealing of target sequences in so called DNA micro arrays. Furthermore, once a target sequence and probe have hybridized and formed a DNA double strand it can be used to start amplification reactions by polymerases to synthesize complementary copies of the target sequence. With the appropriate opposite primer sequences polymer chain reactions (PCR) can be carried out [Saiki, 1985]. Here, the specific sequences of the first and second primer are used to amplify exponentially the entire or partial target sequence. Those amplification products can be used to generate the detection signal either by labeling during the formation by e.g. fluorescence tags, or through subsequent analysis steps, e.g. transferring the product onto a chromatography gel. Primarily, PCR is a fluid phase reaction in which the two primers, target molecules, dNTPs, polymerases and ions need to react. Such reaction of multiple components makes it inherent difficult to be carried on solid supports. Both primers are immobilized in close proximity, which means in molecular dimensions. One solution to the problem has been presented by grafting surfaces with mixed primers. The immobilization of primers, their accessibility, and subsequent use in PCR have been described as solid phase PCR (SP-PCR), asymmetric SP-PCR, bridge PCR and ESP-PCR [Adessi, 2000; Kahn, 2008]. They are mechanistically limited with respect to amplification efficiency and solid support primer involvement a bridge-PCR process. The PCR on such single solid supports shows low efficiencies of 0.15 compared to values close to 1 in fluid phase systems. Reasons are the two dimensional geometric growth of seeded islands, and the unidirectional perpendicular to surface diffusing reaction compounds.

Alternatively, two different primers can be presented by two different surfaces. Opposite and each other facing surfaces and the imposed directionality are inherently consistent with the working principle of the classical PCR scheme in which two primers bind onto opposite locations in or at the end of a target molecules, and in which the direction of the polymerase catalyzed amplification reaction proceeds in opposite direction. Because the probes of the different surfaces need to interact with the same target molecule it is necessary to bring both surfaces into submicron proximity. The primers should be immobilized either after or before the aligning of the two surfaces. The immobilization afterwards can be achieved by electrochemical stripping as published in WO/2010/104479, which is a long multistep process of limited applicability. Otherwise, the spatial resolution and geometry of two opposite surfaces makes conventional primer deposition methods like spotting and piezo-electric printing which produces feature limits of more than 50 μm, photolithographic deprotection which reaches feature sizes of below 10 μm, or photoresist lithography, pWP, pCP/pFN which enables feature sizes of below 1 μm, absolutely unsuitable. If the individual surfaces become modified before the assembly of the two-surface structure, any of the deposition methods mentioned above can be used. However, the challenge is of how to align two surfaces into submicron proximity. The two solid surfaces cannot touch entirely across large areas and should leave access to the fluid phase. It is possible to realize such assemblies in small scales with liquid media atomic force microscopes (AFM), scanning tunneling microscopes (STM) or electrochemical scanning microscopes (ESCM) which are able to control the distance of two surfaces in molecular dimensions. Multi-tip STMs are sophisticated instruments built for structural analysis. Such techniques are not suitable for the design of complex two surface sensor arrays.

One solution to the problem has been found in the design of impedimetric sensor arrays which contain numerous dual solid phase nano-gap junctions. Here, the main structural requirements are that small electrodes are electrically insulated with an ultrathin film which is functionalized with sensor molecules, and that those electrodes approach each other to said submicron distances. Those distances are determined by spacers, which are integrated into the individual surface structures as published in [PCT/AT2012/000043]. Such spacers are made during the multistep etching process and controlled by photolithography, the etching conditions and metal track depositions. Because of the close proximity of their surfaces those structures have the disadvantage that any fluid exchange is dramatically slowed down and that it is inherently difficult to design and execute the microfluidics efficiently. This matter constitutes a significant problem. Volumes of the fluidic phase inside the sensor array cannot be varied.

### Flexible Reaction Chambers, Flexible Biosensors and Lab-on-a-Foil Devices

Flexible biosensors are well known because many analytical test strips for glucose etc. are built on cheap, thin carrier substrates. Such test strips can be produced on fibrous materials like paper or thin polymer films or foils (foils being semi-finished parts of any material which is thinner than 500 μm).

Fluidic systems have been developed which use the properties of flexible foils to design reaction cassettes and integrated microfluidic devices in which fluid transport can easily be achieved in small volumes [US5863502A1]. Such Lab-on-a-Foil systems are characterized by low material consumption of foils, films, sheets, laminates or tapes, low cost materials, and cost-effective high volume fabrication processes.

Based on such integrated foil approach, the Fraunhofer Institute for Modulare Festkörper-Technologien EMFT (Munich, DE) developed a polymer microfluidic D-dimer biosensor system. D-dimer is a molecular marker for the presence of thromboembolytic events. The biosensor measures impedance changes with interdigitated microelectrodes which are caused by immunological binding of D-dimer to specific recombinant antibodies. The antibodies are covalently immobilised on interdigitated electrodes which are located on a single surface. The microfluidic cartridge consists of microfluidic channels which have been crafted in polycarbonate and which are joined to electrodes on a polyethylenenaphthalate foil.

Thin flexible foils have enabled in addition to utilize various features like low thermal resistance for efficient thermocycling. This is of particular use for PCR which is a popular method to amplify specific genetic sequences. In conventional thermocycling devices one PCR cycle takes approximately 60 to 180 sec. Motorola Inc. had developed an integrated laminate-based polycarbonate foil device for DNA amplification and subsequent hybridization [Liu, 2002]. Cooling and heating were accomplished with a Peltier element reaching fast heating rates of 7.9° C.s⁻¹ and cooling rates of up to 4.6° C.s⁻¹. Systems with thinner foils can reach even faster cycling speeds of up to 88 seconds per cycle which enables 40 amplification cycles in just 30 minutes [Jia, 2007]. Another approaches employed circular shaped foils disk for real-time PCR experiments [Focke, 2010], so-called array tapes for PCR-based assays to detect single nucleotide polymorphism. The array tape contains embossed microwells on continuous tape which passes through different stations of pipetting, drying, sealing etc. [U.S. Pat. No. 6,878,345].

All examples utilize individual features of flexible foils. Examples have been given where foil devices are used in PCR applications, hybridization and binding reactions with single surface bound sensor compounds.

### Objective of the Invention

The main objective was to overcome the above mentioned drawbacks in detection of molecules by finding a solution for biosensor arrays where in molecular dimensions reactions between three compounds—a class of first sensor molecules, a class of second sensor molecules and analyte molecules—are facilitated, wherein the first and second class of sensor molecules are separated, so that a predefined gap is formed between them, and a first portion of specific analyte molecules binds to the first sensor molecules and second portions of specific analyte molecules binds to the second sensor molecules. Both portions of the analyte molecules should be able to bind simultaneously to a first or a second sensor molecule. These requirements imply that the sensor molecules are separated only by molecular distances of less than one micrometer which is determined by the length of the analyte molecules. The two solid phases and the one fluid phase form a 3-phase region which needs to be maximized in size to increase the sensor functionality. In addition, the fluxes and exchange rates of the analyte molecules between the inner volume phase and the 3-phase region are maximized.

### SUMMARY OF THE INVENTION

The invention relates to a sensor carrier comprising a flexible film comprising a plurality of functionalized sensor elements each comprising a functional layer, wherein the functional layers are located on the same surface of the film within a window area, being preferably arranged in the centre of the film, wherein a region of each of the functional layers of the sensor elements is functionalized with one or more sensor compounds.

The sensor carrier according to the invention enables to assemble and to form a sensor carrier with flexible films which are functionalized on their respective surfaces. In particular the invention relates to flexible films which contain immobilized surface with exposed sensor biomolecules in dedicated spatial locations. The sensor molecules such as biomolecules are able to react with analyte molecules of a solution. Specific reactions, like the hybridizations of polynucleic acids with shorter oligonucleic acids, lead to signals which are related to the spatial location and quantity of the immobilized sensor molecules. By these means a large number of different reactions can be carried out simultaneously on the surface of a volume and related to the presence and concentration of molecules in the solution. The main advantage of the present invention is the flexibility of the functionalized carrier substrate which enhances the functionality of the sensor and provides additional novel features in contrast to the use of inflexible and stiff carrier substrates.

First sensor molecules are arranged on the first sensor carrier and second sensor molecules are arranged on a second sensor carrier. As to the flexibility of the sensor carriers, a central volume for the analyte can be formed between the window areas of two sensor carriers. Consequently, a reaction as to the objective of the invention is facilitated by the invention.

The flexibility of the film allows movements of the window area relative to the surrounding area.

According to a preferred aspect of the invention it is provided, that the film material has a flexural modulus from 0.5 GPa to 2 GPa, preferably from 0.75 GPa to 1.25 GPa, and/or wherein the thickness of the film is between 10 μm to 300 μm, in particular between 100 μm to 200 μm, and/or wherein the film is composed of a polymer, such as polyethylene, polyethylene terephtalate (PET), polyimide, polyethylene naphthalate, polycarbonate, polyether sulphone, polyarylate and/or mixtures thereof

The thickness of the volume between the sensor carriers, i.e. the distance between the sensor carriers, may be modified easily by the application of relatively low external forces, e.g. by tips applied on the surface of the sensor carriers opposing the central volume, without damaging of the sensor carriers.

For the same reason, it can be alternatively or additionally provided, that the flexibility and elasticity of the material of the film, and/or the flexibility and elasticity of the material of the functional layers, and/or the size and/or shape of the window area, are chosen so that, when the boundary of the window area is fixed in direction normal to the plane of the film, and the window area is deflected and/or deformed, out of the plane of the film in the direction normal to the plane of the film, wherein at least one part of the surface is pushed by 10 to 500 μm, preferably by 20 to 200 μm, into the direction normal to the plane of the film, the film is deflected elastically, in particular without being damaged.

In order to have additional visual feedback of the reaction and to be able to apply radiation of light to the analyte molecules, it can be provided, that at least the window area of the film is transparent.

To obtain a sensor carrier which enables a preferable exchange rate of the analyte molecules and a favourable accumulation of analyte molecules to the sensor carrier, it can be provided, that one or more spacers, preferably each having the form of a protrusion, are arranged on the surface of the sensor carrier on the side of the functional layers, wherein the spacers are elevated of the surface of the sensor carrier by 0.05 μm to 1 μm, preferably by 0.2 μm to 0.5 μm.

To obtain sensors for the detection of biomolecules, it can be provided, that the sensor compounds contain oligonucleotides binding to binding sites of analyte molecules, preferably organic polymers or DNA or RNA molecules.

To obtain a sensor carrier whose single sensors can be easily addressed electronically, a sensor carrier can be provided comprising a plurality of electrical conductors, preferably comprising at least one line shaped portion, wherein each of the conductors is assigned to one sensor element, and each conductor, preferably the line shaped portion thereof, is located underneath the functional layer of the respective sensor element in or on the film, and/or wherein the sensor carrier preferably comprises an electrical connector, which is arranged on the film, the electrical connector comprising a number of connector pads, each of the connector pads being connected to one of the conductors, and/or wherein the conductors in or on the film contain or consist of nickel, copper, gold, carbon, indium tin oxide, semiconductors or any other similar conducting material.

To obtain an easily producible sensor carrier, it can be provided, that the film has a rectangular shape, wherein the conductors are arranged in parallel in at least the window area of the film, and wherein at least one portion of each sensor element and/or each conductor, preferably the respective functionalized region of the sensor element, is inclined to the edges of the film, preferably by an angle of 40° to 50°, especially 45°.

The invention further relates to a sensor arrangement comprising a sensor carrier according to the invention, a gasket comprising a central opening, and a second film with a plurality of functionalized sensor elements each being formed by a functional layer, wherein the functional layers are located on the same surface of the second film within a window area, wherein a region of each of the functional layers of the sensor elements is functionalized, and wherein one or more sensor compounds are arranged or located in the respective functionalized regions of the sensor elements, said second film preferably having a plurality of line shaped electrical conductors and one electrical connector and/or with spacers arranged on its surface, wherein the gasket is arranged between the sensor carrier and the second film, so that a chamber is formed between the sensor carrier and the second film, and the chamber is surrounded by the inner edge of the gasket. With such an arrangement a fully functional sensor, that facilitates the detection of molecules, can be obtained. This arrangement has a preferable design for biosensor arrays where molecular dimensions reactions between three compounds—a class of first sensor molecules, a class of second sensor molecules and analyte molecules—is facilitated. The volume between the sensor carrier and the second film can be easily adopted to the analyte molecules.

A simple arrangement which comprises only one flexible sensor carrier is provided, wherein material of the second film has a flexural modulus higher than 5 GPa. Such a second film may also have the same features as the sensor carrier according to the invention, however the second film need not necessarily have the feature of a high flexibility.

In order to obtain a sensor arrangement with a high density of sensors, it a sensor arrangement can be provided, comprising two sensor carriers according to the invention and a gasket comprising a central opening, wherein the gasket is arranged between the two sensor carriers, so that a chamber is formed between the two sensor carriers and surrounded by the inner edge of the gasket, and wherein the respective surfaces of the sensor carriers, on which the sensor elements are arranged, are facing each other and each sensor element and/or conductor of one of the sensor carriers opposes or faces at least one, preferably each, sensor element and/or conductor of the opposite sensor carrier.

To easily adjust the thickness of the volume by applying pressure with one single tip on the flexible sensor carrier, it can be provided, that particles or beads are arranged between the sensitive opposing surfaces of the sensor carriers within the chamber, wherein the particles or beads are preferably included or dispersed in the analyte fluid containing analyte molecules to by analyzed, wherein the minimum distance between the sensor surfaces of the two films is defined by the diameter of the particles or beads.

In order to obtain a sensor that can be easily filled with an analyte fluid, the sensor arrangement may have an inlet and an outlet leading through one or both of the sensor carriers and/or in the gasket, leading from the outside to the chamber and enabling fluid to stream into the chamber or out of the chamber through the inlet and the outlet.

Furthermore, the invention relates to a cartridge for holding an arrangement according to the invention. Such cartridge comprises an arrangement according to the invention and at least one, preferably two, holder elements wherein the gasket and the sensor carriers or one sensor carrier and one film are fixed and compressed by the holder elements so that the chamber is confined by the gasket, and/or the sensor carriers and/or the film, so that the chamber is sealed and impermeable to liquid. Such cartridge eases handling the sensor arrangement.

In order to obtain a cartridge with which a sensor arrangement can be easily applied a force from one side to set the distance between the films and thickness of the volume and heat from the other side to set the temperature of the fluid within the volume and the temperature of the sensor carriers, it can be provided, that the holder element has at least one opening, wherein the opening is covered by one part of one of the sensor carriers, preferably by the window area, said part at least partially confining the chamber.

To hold a sensor arrangement which enables a preferable exchange rate of the analyte molecules and a favourable accumulation of analyte molecules to the sensor carrier, it can be provided, that the holder element has two opposing openings, wherein the first opening is covered by one portion of one of the sensor carriers, which is preferably the window area of said sensor carrier, and wherein the second opening is covered by one portion of the other sensor carrier or film, wherein the second opening is preferably covered by the window area of the other sensor carrier or film, said portions of the sensor carriers or film at least partially confining the chamber.

In order to obtain a cartridge with which a sensor arrangement can be easily applied a force to set the distance between the films and the thickness of the volume, it can be provided, that the holder element further comprises at least two channels, wherein the first channel connects the inlet with the outer surface of the holder element and the second channel connects the outlet with the outer surface of the holder element.

To avoid the sensor carriers or films of the sensor arrangement from shear movement, it can be provided, that the cartridge comprises two holder elements, wherein

a) the sensor carriers and the gasket, or
b) the sensor carrier and one film and the gasket,
are fixed between the holder elements,
wherein the holder elements preferably comprise protrusions, particularly positioning pins, and holes, matching each other when the holder elements are assembled, and
wherein the sensor carriers, the film and/or the gasket have positioning holes being penetrated by at least some of the protrusions.

To seal the chamber, it can be provided, that the cartridge comprises two holder elements, the holder elements compressing the arrangement to seal the chamber.

It is a further objective to determine the amount of a specific analyte compounds in an analyte fluid, where only analyte compounds of a specific molecular length can be determined.

The invention solves this objective with a method according to the invention. The invention relates to a method for the measurement of a concentration of the analyte compound in an analyte fluid, preferably with two sensor carriers or a sensor arrangement or a cartridge according to one of the preceding claims,

a) wherein an elastically flexible first film and a, preferably elastically flexible, second film are provided, a first sensor compound being arranged on a region of the first film and a second sensor compound being arranged on a region of the second film, the sensor compounds being arranged on opposing sensor surfaces of the films facing each other,
b) wherein the first sensor compound binds or adheres to a first portion of the molecule of the analyte compound and the second sensor compound binds or adheres to a second portion of the molecule of the analyte compound, and
c) wherein the analyte fluid is arranged in a chamber formed between and surrounded by the two films,
d) wherein the first film and the second film are elastically deflected or deformed in a manner, that the distance between the opposing surfaces of the films facing each other is reduced and equals or is smaller than the distance between the first portion and the second portion of the molecule of the analyte compound,
e) so that a sensor is formed between the region of the first film on which the first sensor compound is arranged and the region of the second film on which the second sensor compound is arranged, where said regions are arranged to face each other, and
f) wherein the amount of molecules bound or adhered to the first and second sensor compounds is measured.

In order to determine the distance between the films, it can be provided, that particles or beads of a given diameter are added to the analyte fluid, wherein the particles or beads are filled into the chamber between the two films, so that the minimum distance between the sensor surfaces of the two films is defined by the diameter of the particles.

In order to automatically determine the amount of the analyte compound in the analyte fluid, it can be provided, that the capacitance or impedance between the electrical conductors in the regions of the first film and the second film on which sensor compounds are arranged is measured, and said capacitance or impedance indicating the amount of the analyte compound in the analyte fluid.

To determine the amount of a plurality of different analyte compounds in parallel, with two sensor carriers according to the invention are used, each of which comprises one film or with an arrangement according the invention or a cartridge according to the imvention comprising the films, wherein a plurality of sensors is formed between the regions of the first film on which the first sensor compound or plurality thereof is arranged and the regions of the second film on which the second sensor compound or plurality thereof is arranged, where said regions are arranged to face each other, and wherein the concentration of analyte compounds located or deposited between two facing regions of a sensor is determined separately for each sensor.

In order to control the amount of analyte fluid in the chamber, it can be provided, that before setting the distance of the opposing films the distance of the films is increased in order to increase the amount of fluid within the chamber and/or the distance of the films is regulated to adjust the internal volume of the chamber in order to control the amount of analyte fluid accommodated in the chamber.

In order to increase or decrease the velocity of the analyte compounds in the analyte fluid and to remove partially bound analyte compounds from the sensor compounds of the films, it can be provided, that the distance between the films is, preferably repeatedly, changed before or during the measurement so that the analyte fluid in the chamber is mechanically agitated, so that by adjusting the flow in chamber a specific shear force distribution profile is achieved in the chamber.

In order to fill or empty the chamber before or after measurement, it can be provided, that the distance between the films is changed in order to pump fluid into the chamber or out of the chamber.

### BRIEF DESCRIPTION OF THE DRAWINGS

Some examples of the invention are illustrated in non-limiting manner with reference to the following drawings.

FIG. 1 presents the drawing of an example of a sensor carrier according to the invention in top view.

FIG. 2 illustrates a partial section of the sensor element of FIG. 1 of plane A-A.

FIG. 3 highlights a detail of FIG. 2 with a sensor element with functional coating.

FIG. 4a illustrates a part of the section of plane A-A of an arrangement comprising two sensor carriers as shown in FIG. 1 without external application of pressure, the unloaded state.

FIG. 4b illustrates the arrangement of FIG. 4a where external pressure F is applied via a tip, loaded state.

FIG. 5 shows a cross section of a sensor cartridge comprising the arrangement of FIGS. 4a and 4b.

FIG. 6 displays the cartridge of FIG. 5 in exploded view.

FIG. 7 shows a heating tip.

FIG. 8 illustrates the cartridge with an arrangement of FIG. 5 where external pressure is applied via a tip.

FIGS. 9a and 9b show sectional views of a cartridge of FIG. 6 being equipped with two tips for heating and application of pressure.

FIG. 10a shows a sensor arrangement in the loaded state having a film-gasket-film sandwich structure, where one of the sensor carriers features spacers on its surface.

FIG. 10b illustrates a sensor arrangement in the loaded state having a film-gasket-film sandwich structure, where the films of the sensor carriers are kept separated by particles.

FIG. 10c shows an arrangement having a film-gasket-film sandwich structure, where the flexural rigidity of one of the films is substantially higher than the flexural rigidity of the other film.

FIG. 11 illustrates a section view of reader instrument with a cartridge placed in the mechanical adaptor with a clamping mechanism formed by one upper and one lower arm, and the thermocycling adaptor.

FIG. 12 schematically depicts an overview scheme of the reader instrument.

FIG. 13a shows the impedimetric scanning of an electrode junction array before PCR with a target DNA sequence present in solution.

FIG. 13b shows the impedimetric scanning of an electrode junction array after 40 PCR cycles under otherwise the same conditions.

The mean and standard deviation of the differences in the impedance modulus of identical junctions with respect to their functionalization are shown in FIG. 14. The functionalizations are encoded with F, forward primer; R, reverse primer; N, non matching primer; and 0, no primer.

### DETAILED DESCRIPTION OF PREFERRED EMBODIMENTS

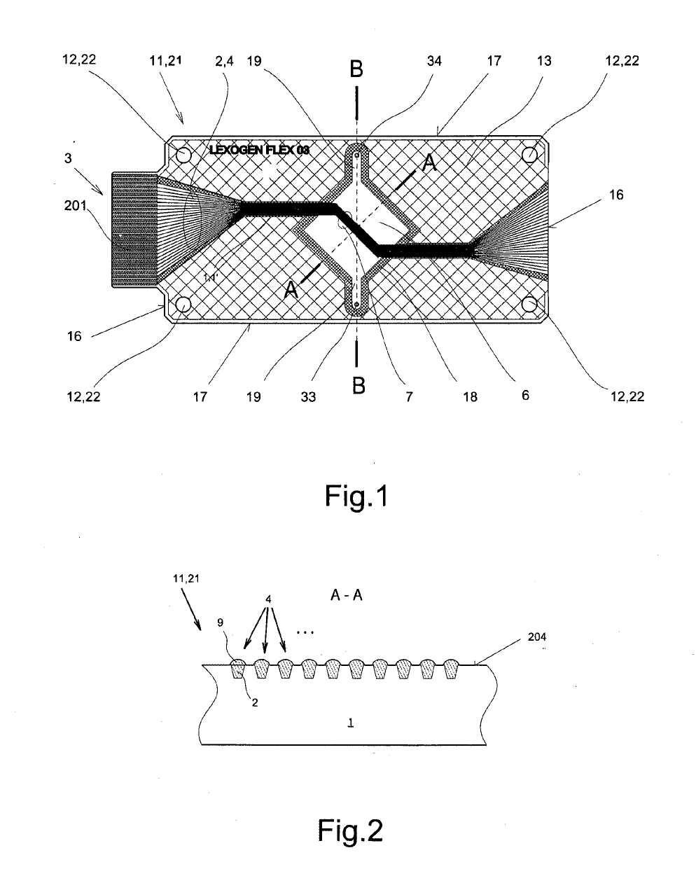

A sensor carrier 11 according to a first example of the invention is depicted in FIG. 1. The sensor carrier 11 comprises a flexible film 1 with twenty four functionalized sensor elements 4. The base film 1 of the sensor carrier is made of a heat stabilized polyethylene terephthalate (PET), such as MELINEX ST504 (DuPont Teijin Films, USA).

Typically, the film 1 can be made of any material which is not permeable for the constituents of the solution used in the reaction, is compatible with, i.e. inert with respect to, the reaction, and withstands the temperature changes required for PCR thermocycling under mechanical load without permanent deformations. Optimally, the material has a good thermal conductance, so the rapid temperature changes of the analyte in the chamber 32, i.e. on the sensor side 204, 204′ of the film 1, can be achieved when the film is cooled or heated on its non-sensor side 205, 205′. It is of advantage to use a transparent material if optical readout with light excitation and emission detection should be carried out through the film. From the point of view of the raw biosensor production technology, materials such as Polyimide (PI), Polyethylene Naphthlate (PEN), Polyethylene Terephthalate (PET), Polycarbonate (PC), Polyether Sulphone (PES), Polyarylate and other can be used.

The film 1 is typically produced by technologies used for flexible circuit boards (FCB) or similar, combined with chemical technologies used for coating the film surface with insulating layers and with layers exposing the immobilized reaction partners, referred to as 5′-end and 3′-end primers, or also referred to as forward (F) and reverse (R) primers. It is particularly suitable to produce the films 1 using the ultra-high resolution flexible circuit boards technology. Such technologies are known in the art and have been disclosed, e.g., in the document U.S. Pat. No. 6,284,072. The advantages offered by these technologies include very small structures, down to 3 μm track width and 5 μm gap width, and nearly planar surfaces. Unlike the standard FCB manufacturing process, resulting in polymer substrates with electrical conductors 2 with typical height of 10 to 50 μm over the base surface, the said ultra-high resolution FCB technology produces electrical conductors 2 formed by metallic tracks which are embedded in the substrate, so that resulting surface is planar with roughness 5 to 15 nm (rms). The tracks of the electrical conductors 2 are typically made of nickel with gold coating.

The material of the film 1 has an upper temperature limit of 140° C. and good optical and mechanical properties. The Young's modulus of the material of the film 1 is 2.0 to 2.7 GPa. The ultimate tensile strength of the material of the film 1 is 55 MPa. Poisson's ratio of the material of the film 1 is between 0.37 to 0.44. Thermal conductivity of the material is 0.15 to 0.40 W·m⁻¹·K⁻¹. Thickness of the film 1 is 125 μm. The flexural modulus of the material of the film 1 is 1 GPa. The flexural modulus of the film can for instance be determined by the ASTM D 790 standard.

As shown in FIG. 2 the film 1 has recesses in which electrical conductors 2 are embedded to form conductive tracks. These tracks or conductors 2 are manufactured using specific technology for ultra-high density flexible circuit boards. The electrical conductors 2 are made of nickel with a thickness of 5.4 μm, which are coated with a gold layer of 0.1 to 0.2 μm thickness. The surface of the electrical conductors 2 is almost isoplanar to the surrounding PET surface, with a height difference of up to 100 nm. The overall thickness of the film 1 including the layer with embedded electrical conductors 2 is 145 μm.

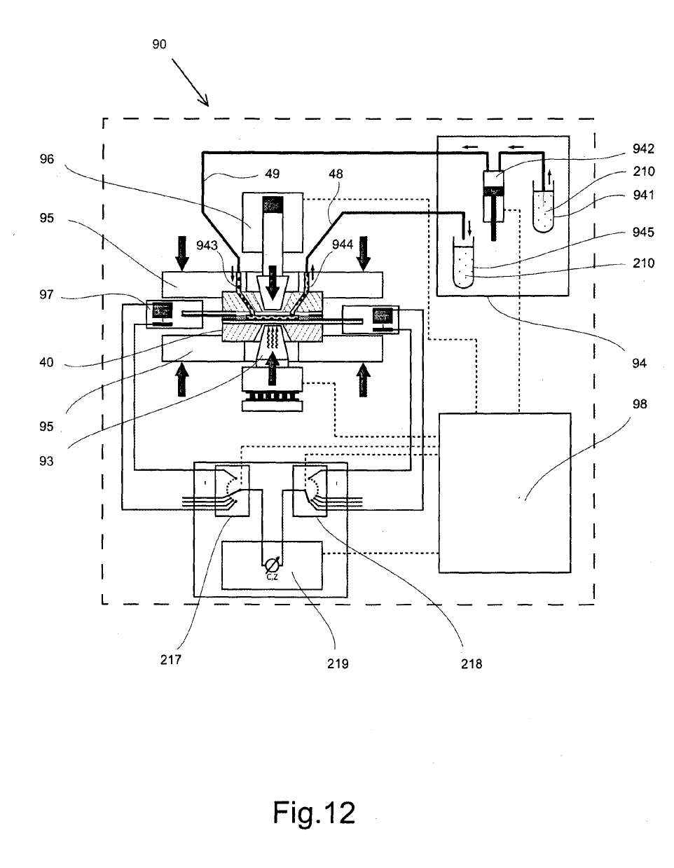

In the first example of the invention, the film is rectangular with an outline of 65×25 mm. The outer dimensions of the film allow to position the film into a standard microarray scanner and to scan it similarly to a microarray on a 25×75 mm glass carrier. It is also a convenient size for inspection using optical microscopy. The electrical conductors 2 of the sensor elements 4 are connected to an electrical connector 3 comprising a plurality of connector pads 201, wherein typically, each sensor element 4 is connected to a single connector pad 201. The electrical connector 3 is located typically in the marginal area of the sensor carrier 11, 21. On both shorter edges 16, the film 1 comprises electrical connectors 3 which are in this very example formed as tongues having a width of 15.5 mm and a length of 7 mm. The form and arrangement of electrical connectors 3 and connector pads 201 match a standard industrial 30 way, 0.5 mm pitch FCB connector. This electrical connector 3 connects the conductors 2 of the sensor carrier 11, 21 with external electronics circuits 219 located in the readout instrument 90 (FIG. 12). Note that in FIG. 1, the electrical connector 3 is only shown on one shorter edge 16, as the electrical connector 3 on the other shorter edge 16 is typically cut off before application (see below).

The film 1 has twenty four electrical conductors 2 in the form of partly linear tracks, leading from one electrical connector 3 located on one short edge 16 of the film 1, via a window area 6 of the film 1, to the other electrical connector 3 on the opposite short edge 16 of the film 1. In the window area 6 the electrical conductors 2 are linear and parallel to each other. The conductors 2 are 25 μm wide, and aligned in a 50 μm pitch.

In the window area 6 the angle between the short edges 16 and the long edges 17 of the film 1 and the electrical conductors 2 is 45°.

The electrical conductors 2 are connected to the connector pads 201 in the center of the 30-way connectors 3 located on both sides of the film 1. The six remaining outer connector pads 201 are shorted and electrically connected to a shielding 13.

The film 1 of the sensor carriers 11, 21 has four positioning holes 12 with a diameter of 2 mm each in the four corners, and two fluidic openings 33, 34 with a diameter of 0.5 mm, forming an inlet 33 and an outlet 34 of a sensor arrangement 207.

The window area 6 of the film 1 has a squared shape which is surrounded by a diamond shaped shielding element 18. The edges of the shielding element 18 and the edges of the film 1 are rotated by 45°. Two corners of the diamond shaped shielding element 18 which are near the longer edges 17 surround two channels 19, where each of the channels 19 contains one fluidic opening 33, 34.

The remaining area of the film 1 outside the window area 6 which is not covered by the electrical conductors 2, connector pads 3 or positioning holes 12 is covered with a hatching pattern shielding 13 consisting of further electrical conductors. The conductors of that hatching pattern shielding 13 are arranged for technological reasons to avoid curling of the film 1. This hatching pattern shielding 13 is connected to the shielding element 18 of the window area 6 and the six unused connector pads 15 of the electrical connectors 3.

Immediately after production, the film 1 has two tongue shaped connectors 3 on which connector pads 201 are arranged.

For quality control purposes, the electrical conductors 2 of the sensor elements 4 lead from the electrical connector 3 on one short edge of the film 1 via the window area 6 of the film 1 to another secondary electrical conductor 3 on the opposing short edge of the film 1, allowing to check the integrity of the electrical conductors 2. In order to quality control the fabrication of the conductors 2, both electrical connectors 3 are attached to an external electrical circuit, wherein the resistance of each electrical conductor 2 can be measured. Possible short connections between the electrical conductors 2 and/or broken electrical conductors 2 can be detected. A typical break detection method involves connecting the two corresponding connector pads 201, 201′ of one electrical conductor 2 to an electrical circuit and measuring the resistance which should ideally be close to zero Ohm. A typical short detection method consists in measuring the resistance between any neighboring two electrical conductors 2, which should ideally be infinitely high. After this quality check, one of the tongue-shaped electrical connectors 3 may be cut off.

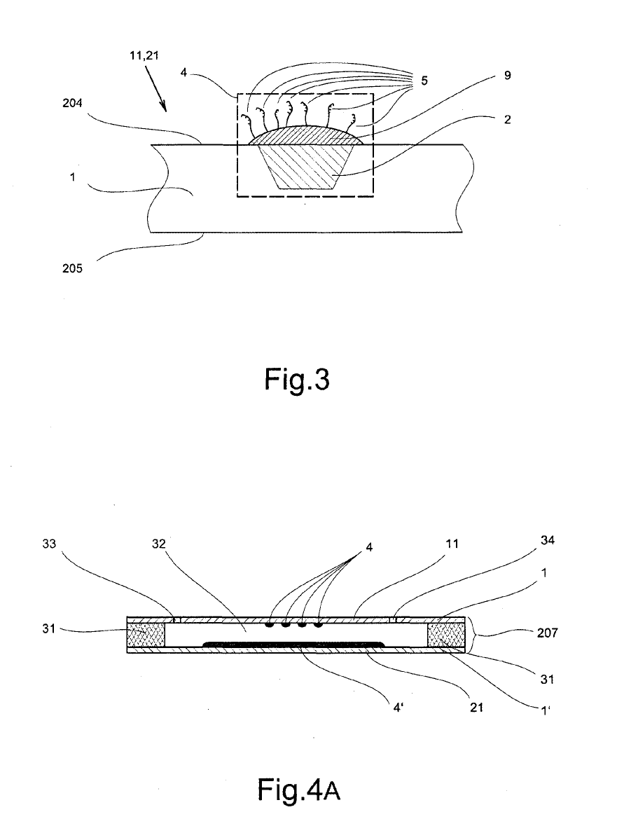

In a next production step the films 1 of the sensor carriers 11, 21 are functionalized by a specific electrochemical process. FIG. 3 shows a detailed section view through the sensor carrier 11, 21 with a sensor element 4 comprising a functionalization coating or layer 9 on top of an electrical conductor 2. It is not necessary that the electrical conductor 2 is fully covered by a functionalization layer 9, moreover it is possible that only smaller regions 7 located directly above the electrical conductor 2 are functionalized and covered with a functionalization layer 9. Preferably all the functionalized regions 7 are located in the window area 6 of the sensor carrier 11, 21.

In order to form a functionalization layer 9, sensor compounds 5 are immobilized through the functionalization coating 9 which produces the layer 7 of the sensor element 4. The sensor compounds 5 are exposed on the sensor surface 204 of the sensor carrier 11, 21.

Each of the electrical conductors 2 may be coated with an individual insulating functionalization layer 9 or coating comprising an individual sensor compound 5. In this very example, the sensor compound 5 comprises immobilized oligonucleotides which are exposed to the surface of the electrically insulating functionalization layer 9.

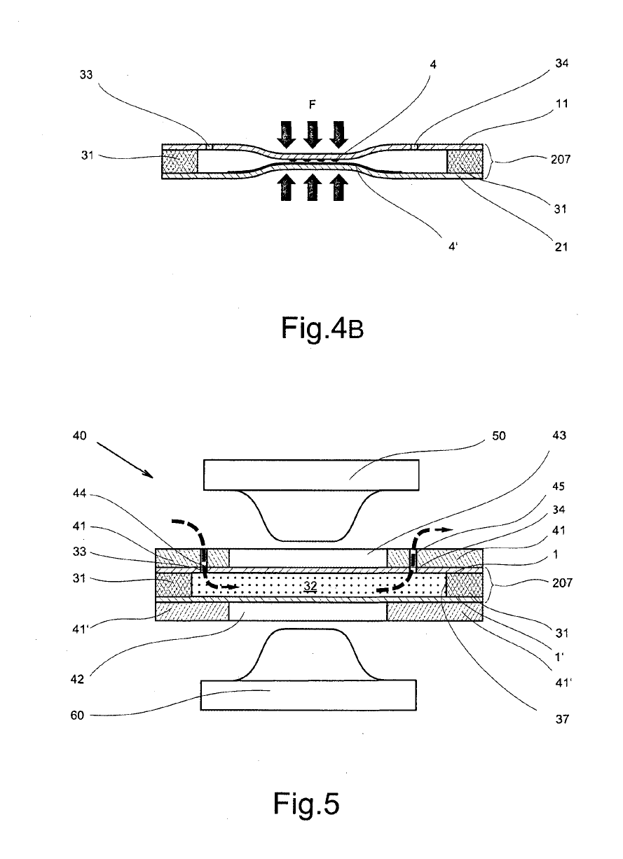

FIG. 4a illustrates a sensor arrangement 207 of two sensor carriers 11, 21 as shown above forming a sandwich structure, where a gasket 31 is placed between the sensor carriers 11, 21. As already mentioned, both sensor carriers 11, 21 have the same dimensions and are made of polyethylene terephthalate (PET). The sensor carriers 11, 21 comprise a flexible film, which can easily be deformed, i.e. bended and flexed by hand. A series of linear sensor elements 4, i.e. electrical conductors 2, up to 1000 per film 1, typically possessing a width of several microns to several tens of microns, are arranged in a parallel pattern on the sensor carriers 11, 21. In this first example the films contains 24 electrical conductors 2. Each of the sensor elements 4 comprises an electrical conductor 2 and a functionalized layer 9 or coating. The electrical conductor 2 is covered with the functionalized layer 9 or coating in at least one surface region 7 of the film 1, which is usually located in the window area 6 of the film 1. The electrical conductor 2 are realized on the same side of the film 1.

Typically an arrangement 207 is formed in a sandwich shape comprising the following layers: a first sensor carrier 11, a gasket 31 being arranged on the sensitive sensor surface 204 of the sensor carrier 11, the gasket 31 comprising a central opening 38 matching the window area 6 of the first sensor carrier 11, and a second sensor carrier 21 with the sensitive sensor surface 204 facing towards the gasket 31 and the sensitive sensor surface 204 of the first sensor carrier 11. The two sensor carriers 11, 21 of the arrangement 207 are geometrically identical, where the functionalized coatings or layers 9 of the single sensor elements 4 may contain different sensor compounds 5.

In this very example, the arrangement 207 comprises two sensor carriers 11, 21 with identical geometry. The form and size factor of the chip are chosen to be compatible with the standard sample platforms used in molecular biology. This allows using the sensor carriers in combination with different sample preparation and detection/analysis technologies used in this scientific domain, such as microarray spotting, microarray scanning, optical and other microscopy etc.

The gasket 31 of the arrangement 207 is typically made of elastic material and has an outline form and a size corresponding to the film sensor carriers 11, 21, with exception of the area of the connector pads 201 of the film. The typical thickness of the gasket 31 is from 100 to 250 μm. In this example, the gasket 31 consists of a polytetrafluoroethylene (PTFE) or Teflon film (Polytetra, Germany) with a thickness of 250 μm.

The outer edge of the gasket 31 matches the outline of the sensor carriers 11, 21, wherein the gasket 31 does not cover the electrical connectors 3. The gasket 31 has four holes matching the positioning holes 12 of the sensor carriers 11, 21.

The areas of the connector pads 201 area of both films 1, 1′ stand out of or project from the assembly and are accessible for electrical connection. Thus, all the sensor elements 4 of sensor carriers 11, 21 are electrically accessible for impedance and/or capacitance readout.

The gasket 31 has a central opening 38, typically of a square or round shape, which covers the window area 6 of the film when the outlines of the film 1 and the gasket 31 are aligned to each other. The central opening 38 of the gasket may be slightly larger with respect to both planar directions than the window area 6 of the film 1. This opening 38 is arranged so that, when the gasket 31 and the film 1 are aligned, it also covers the two fluidic openings 33, 34 of the film 1. In this very example, the inner boundary edge of the gasket 31 matches the shielding 18 of the window area 6, so that consequently the opening 38 of the gasket 31 is connected to the inlet 33 and the outlet 34.

The gasket 31 also has positioning holes 39, which, when the gasket 31 and the sensor carriers 11, 21 are aligned, coincide with the positioning holes 12 of the film 1. The form of the gasket 31 allows the area of the connector pads 201 of the film 1 to protrude from the gasket 31 and remain accessible for an external connector, even when the sensor carrier 11, 21 is arranged in a sensor arrangement 207.

When the said sandwich arrangement 207 of a first sensor carrier 11, a gasket 31, and a second sensor 21 carrier is assembled, a chamber 32 or compartment is formed between the gasket 31 and the sensor carriers 11, 21. The volume of the chamber 32 is confined by a part of the sensor side surface 204 of the first film 1, by the part of the sensor side surface 204′ of the second film 1′, and by the side of the inner edge of the gasket 31.

The window areas 6 of the sensor carriers 11, 21 are exposed to this chamber 32. The chamber 32 is fluidically connected with the non-sensor surface 205 of the first sensor carrier 11 opposing the sensor surface 204 and the non-sensor surface 205′ opposing the sensor surface 204′ of the second sensor carrier 21 by the fluidic openings 33, 34 in the films 1 of the sensor carriers 11, 21.

In order to obtain a flow of a fluid through the chamber 32 formed between the sensor carriers 11, 21 and the gasket 31 there is at least one inlet 33 and one outlet 34. It is possible that both inlet 33 and outlet 34 are located in one of the sensor carriers 11, 21. It is however also possible that the inlet 33 is located in one and the outlet 34 is located in the other of the sensor carriers 11, 21.

Usually, only two of the four fluidic openings 33, 34 and 33′, 34′ in the films of the first and second sensor carrier 11, 21 are used as fluid inlet 33 and outlet 34. The remaining two holes are sealed at the non-sensor surface 205 of one of the sensor carriers 11, 21. Even if the two fluidic openings 33, 34′ are not used, it is of advantage that these fluidic holes are available in the films of both sensor carriers, because there both sensor carriers 11, 21 may be produced by one single unified design.

The film has openings 33, 34 which serve for fluidic connection of the both sides of the film 1. The reaction fluid is located in the chamber 32 which forms a volume compartment, one wall of which being formed by the sensor surface 204 of the film 1. The other side of the film 1, i.e. the non-sensor surface 205 of the film 1, is connected to a fluidic adaptor 94 of the reader instrument 90 as shown in FIG. 12 accommodating the film 1. The fluidic openings 33, 34 are typically positioned near the window area 6 of the film 1, in a symmetrical pattern. Typically, each film 1 has two fluidic openings 33, 34, one of the fluidic openings serving as an inlet 33 and one of the fluidic openings 34 serving as an outlet. Since the arrangement 207 of the holes is symmetrical, their function can be freely exchanged.

To help accurate alignment of the films 1 and 1′ and the gasket 31 to each other, the films 1, 1′ and the gasket 31 also have typically four alignment or positioning holes 12, 22, 39 in the four corners of the rectangular shape. These holes 12, 22, 39 serve as positioning aid, and, in the sensor arrangement, are penetrated by protrusions formed by positioning pins 209 fixed in the external holder element 41.

In order to seal the chamber 32 and to avoid fluid inside the chamber 32 from leaking a further holding force is applied in the region around the circumference of the central opening 38 of the gasket 31. The applied force is directed normal to the films 1 of the sensor carriers 11, 21. Such a force can be applied by magnets.

In the initial state of the sensor arrangement 207 as shown in FIG. 4a the distance between the sensor side surface 204 of the first film 1 and the sensor side surface 204′ of the second film 1′ is given by the thickness of the gasket 31. This distance or clearance allows for convenient filling of the fluid into the chamber 32. Hereby fluid comprising analyte molecules is filled into the chamber 32 via the inlet 33, while the fluid or gas within the chamber 32 is pushed out of the chamber 32 through the outlet 34.

In the window area 6 of the film 1, the sensor elements 4 are arranged in parallel and in such a way that when two identical sensor carriers 11, 21 are brought together and aligned with the electrical conductors 2 and sensor elements 4 sides facing each other, the sensor elements 4 form a mesh, wherein each sensor element 4 of the first sensor carrier 11 faces or opposes each sensor element 4 of the second sensor carrier 22 in at least one portion, referred to as junction. Typically, the films 1, 1′ have a rectangular or similar shape. The sensor element bundle in the window area 6 is arranged at an angle of 45° relative to the sides of the film 1. Thus, when two such sensor carriers 11, 21 are brought together with the outlines aligned, the sensor elements 4 in the window area of the film 1 of the first sensor carrier 11 cross the sensor elements 4 in the window area 6 of the film 1′ of the second sensor carrier 21 at the right angle, forming a rectangular grid of junctions. The window area 6 is then defined by the square including all the junctions of the rectangular grid, the sides of the square being at an angle of 45° relative to the sides of the film outline rectangle. Typically, the centre of this square coincides with the centre of the rectangular outline of the film 1.

In FIG. 4b the application of pressure to the sensor carriers 11, 21 is shown in detail. The sandwich shaped arrangement 207 is compressed by applying compression force F to the window areas 6 of the sensor carriers 11, 21. As shown in FIG. 4b, compression force F is applied on both opposing carriers 11, 21.

Since the elasticity of the material of the films 1, 1′ of the sensor carriers 11, 21 and the thickness of the films 1, 1′ allow for relatively easy elastic deformation with respect to forces and pressures typically applied, the window areas 6 of both sensor carriers 11, 21, which are not in direct contact with the gasket 31, can be elastically deflected in direction towards or away from each other, if suitable forces are applied. Due to this deformation, the volume and the thickness of the chamber 32 decreases, i.e. negative deformation, or increases, i.e. positive deformation, respectively. Consequently, the distance between the sensor side surfaces 204, 204′ of the films of the first and the second sensor carrier 11, 21 in the window area 6 can be controlled by the applied forces.

The distance between the sensor side surfaces 204, 204′ of the films 1, 1′ may be regulated by applying force to the fluid within the chamber 32 and thereby deform the films 1, 1′ so that the distance between these films 1, 1′ is increased. In order to decrease the distance between the films 1, 1′ mechanical force can be applied from outside of the sandwich 207 by compressing the window area 6 of the sandwich 207 from both sides by suitable shaped tips 50, 60 as shown in FIG. 5.

Simultaneous binding or hybridization reaction of analyte molecules inside the chamber 32 and electrical impedance and/or capacitance measurements are dependent on the distance between the sensor side surfaces 204, 204′ of the first 1 and the second film 1′. Therefore setting the distance between the sensor side surfaces 204, 204′ of the films 1, 1′ may be used to adapt the respective sensor arrangement 207 to certain kinds of hybridization reactions for molecules of a given length.

To mechanically hold the sandwich shaped sensor arrangement 207 during manipulation and also to enable a convenient fluidic, electrical, and thermal connection to the reader instrument 90 (FIG. 12), it is of advantage to enclose the said arrangement 207 in a cartridge 40. An example of such a cartridge 40 is shown in FIG. 5. The cartridge 40 comprises two holder elements 41. Each holder element 41 has the form and size to carry the sensor arrangement 207 and is convenient for manipulation. In the example shown in FIG. 5, the cartridge 40 has the shape of a rectangular block of about 50×50×20.5 mm made of a transparent polymer, typically polycarbonate (PC).

The arrangement 207 is fixed between two similar holder elements 41, 41′ and aligned with positioning pins 209, which are attached to one of the holder elements 41, 41′, and passing through the alignment or positioning holes 12, 22, 39 in the first film 1, in the gasket 31, and in the second film 1′. The second holder element 41′ has openings 47 which match the position of the positioning pins 209. The holder elements 41, 41′ also have a mechanical, electromagnetic, magnetic or other similar clamping mechanism, which allows keeping the two holder elements 41, 41′ with the film-gasket-film sandwich 207 together. Typically, the holder elements 41, 41′ have four permanent magnets 214 fixed and aligned around the central opening 42, with suitable magnetic polarity, so two similarly assembled holders elements 41, 41′ are held together by magnetic forces.

Each of the holder elements 41, 41′ has one central opening 42, 43 coinciding with the window area 6 of the sensor carriers 11, 21. Furthermore the holder elements 41 of the cartridge 40 have two fluidic channels 44 and 45 coinciding, on the holder side facing the sensor carriers 11, 21, with the fluidic openings 33, 34 in the film 1.

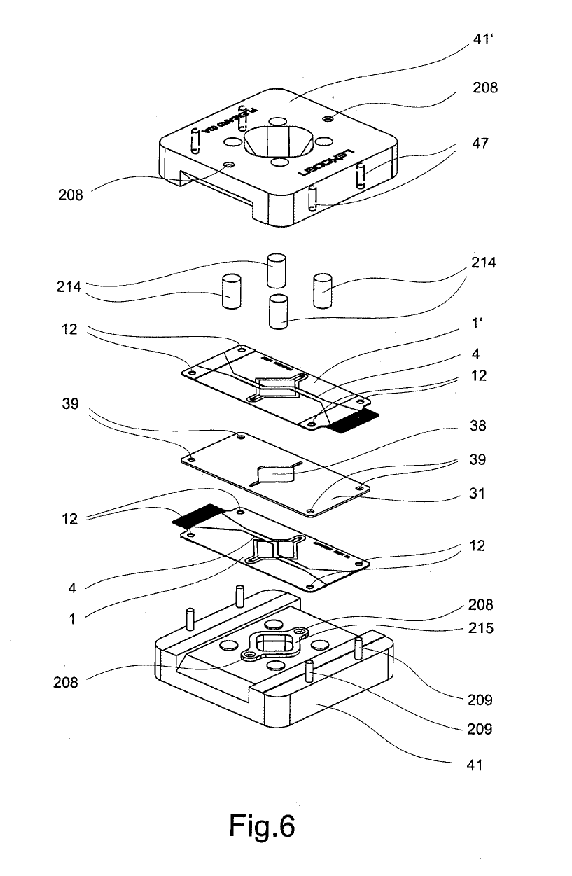

FIG. 6 shows an explosion view of the cartridge 40 formed by two holder elements 41, 41′ and of the sandwich formed by two sensor carriers 11, 21 and a gasket 31. The holder element 41 contains positioning pins 209, which penetrate the positioning holes 12 in the films 1, 1′ of the sensor elements, and the positioning or alignment holes 39 of the gasket 31, and are inserted into the holes 47 in the opposing holder element 41′. The cartridge 40 is held together by magnetic forces exerted by permanent magnets 214 fixed in the holder elements 41, 41′. The sensor carriers 11, 21 and the gasket 31 of the sensor arrangement 207 are compressed by the magnetic forces of the magnets 214, in particular in the rim area 215.

The central opening 42 of the holder element 41 allows access to the window area 6 of one of the films 1, 1′ from the film-gasket-film arrangement 207, so in the full cartridge 40, the window areas 6 of both films 1, 1′ in the arrangement 207 are accessible from opposite sides.

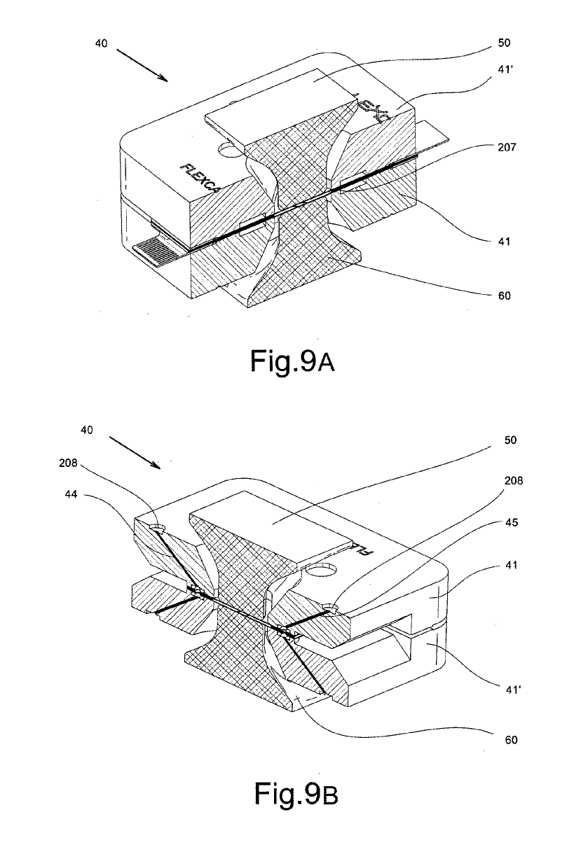

The holder 41 has fluidic channels 44, 45 which coincide with one end (film end) with the fluidic openings 33, 34 in the films, and with the other end (instrument end) with fluidic connection in the reader instrument 90. The reaction fluid or analyte fluid 210 can thus be filled from the reader instrument 90 (FIG. 12) through one of these fluidic channels 44, 45 into the chamber 32 of the sandwich 207. The fluidic channels 44, 45 (FIG. 9a, 9b) may be drilled in the direction normal to the holder side facing the arrangement 207. Alternatively, they can be made in a more complex manner, to adapt the position of the instrument end to the specific design and requirements of the reader instrument 90. Both of the two holder elements 41, 41′ may have the fluidic channels 44, 45 which allows for a single unified design of the holder. In this case, the fluidic channels 44, 45 in one of the holder elements 41, 41′ can be blinded or sealed and not used. Alternatively, there might be two different types of holder elements 41, 41′, one with and another without the fluidic channels, wherein a complete cartridge 40 is always assembled using one holder with fluidic channels and one holder without fluidic channels.

On the side of the holder element 41 which is facing the film 1 is a rim feature 215 matching the size and the form of the window area 6 of the film 1, and in particular, the size and the form of the central opening 38 of the gasket 31. The rim 215, typically 1 to 2 mm wide, is made around the shape of the central opening 38 of the gasket 31. When two holder elements 41, 41′ are assembled to a cartridge 40, the rim 215 of the holder element 41 matches the rim of the opposite holder element 41′, and they both compress the two films 1, 1′ around the margin of the central opening 38 of the gasket 31. Thus, when the two holder elements 41, 41′ are additionally compressed by a force applied in the direction normal to the film-gasket-film sandwich 207, this force allows tight sealing of the chamber 32 in the sandwich 207.

The connections between the sensor carriers 11, 21 and the fluidic channels 44, 45 in the holder elements 41, 41′, and the connection between the channels 44, 45 and fluidic channels 48, 49, in the reader instrument 90 are shown in FIG. 12. The connections are sealed by O-rings which are placed in corresponding pockets 208 (FIG. 9b).

To allow for sealing of the fluidic connection between the fluidic channels 44, 45 in the holder element 41 and the fluidic openings 33, 34 in the film 1, the holder elements 41, 41′ have said pockets 208, i.e. shallow holes, that are concentric with the location of the fluidic openings 33, 34 in the film 1 and the respective ends of the fluidic channels 44, 45. If silicone rubber or similar elastic O-rings of suitable size, with a free state height slightly greater than the depth of the pocket, are placed into the pockets 208, the fluidic connections between the holder elements 41 and the film 1 are sealed. The holder elements 41 can also have similar pockets 208 with O-rings at the instrument side of the fluidic channels 44, 45, when similar fluidic interface is provided by the reader instrument 90.

When assembled, the cartridge 40 has dimensions of 50×50×20.5 mm, the last dimension being the sum of the height of two holder elements 41 and the film-gasket-film arrangement 207. The fluidic channels 44, 45 connect the outer side of the cartridge 40 with the chamber 32 in the arrangement 207. In this very example, only the fluidic openings 33, 34 of the upper holder element 41 are used. The fluidic connections between the lower sensor carrier 11 and the lower holder element 41′ are blinded and sealed by replacing a sealing O-ring with a matching size silicone disc.

The electrical connectors 3 of the sensor carriers 11, 21 are accessible from outside the cartridge, because the connectors 3 have the shape of tongues protruding from the films 1, 1′ of the sensor carriers 11, 21 and extend beyond the cartridge 40. Special zero-insertion force connectors may be used to electrically contact the connectors 3 of the sensor carriers 11, 21 of the cartridge to the reader instrument 90 (FIG. 12).

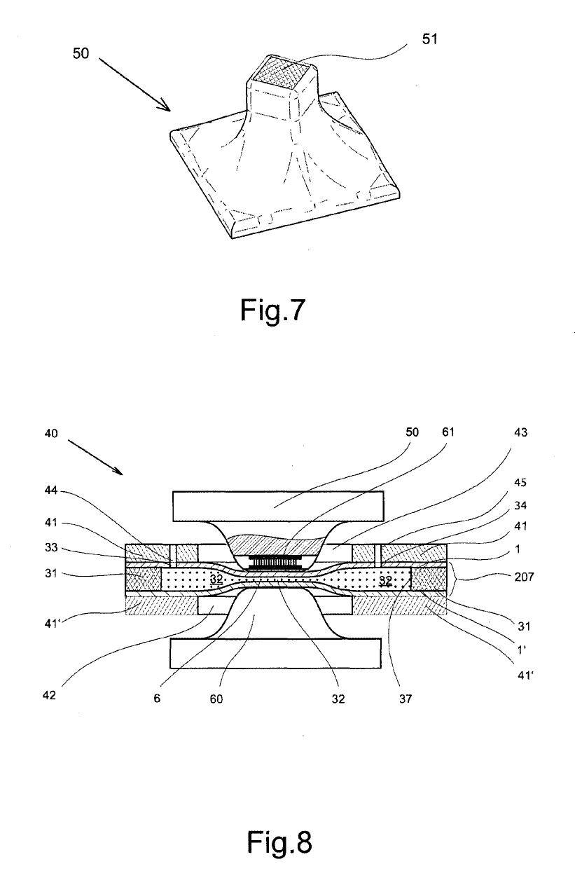

FIG. 7 illustrates the tip 50 of the thermocycling adaptor, shaped in a truncated pyramidal form. The tip 50 is used to penetrate the central opening 42, 43 of the cartridge 40 and apply forces to the sensor carrier 11, 21. The tip 50 touches the window area 6 of the sensor carrier 11, 21 on its contact surface 51. The tip 50 may also contain a thermoelectric element 61 for heating or cooling of the sensor carrier 11, 21 to control the temperature of the fluid within the chamber 32 of the sensor arrangement 207.

FIG. 8 shows a section view of sandwich 207, formed by a first sensor carrier 11, a gasket 31, and a second sensor carrier 21, enclosed in the cartridge 40 formed by two holder elements 41, 41′, and compressed in the window area 6 of the sandwich by two tips 50, 60. Both tips 50, 60 are inserted in the central openings 42, 43 of the holder elements 41, 41′. A chamber 32 is formed between the two films 1, 1′ and the inner edge 37 of the gasket 31. The reaction fluid can be filled into the chamber 32 via the fluidic channels 44, 45 of the holder element, and via the fluidic openings 33, 34 in the films 1, 1′. One of the tips 50, 60 contains a thermoelectric element 61. Due to the material properties and the small thickness of the films, it is possible to thermally affect the analyte fluid 210 inside the chamber 32 by contacting the outer sides of the arrangement in the window area 6 with actively heated and/or cooled tips 50, 60. The thermal energy will be conductively transferred through the film 1, 1′ wall to the fluid within the chamber 32 and vice versa, with the effect focused on the window area 6 of the films only, so rapid local heating and cooling can be achieved. This is preferable for the PCR amplification of the DNA.

In FIG. 9a is shown a section view of the cartridge 40 formed by two holder elements 41, 41′ and the sensor arrangement 207, and the compression of the sensor arrangement 207 by two tips 50, 60.

In FIG. 9b is shown a section view of the cartridge 40 with the fluidic channels 44, 45 in the holder elements 41. Pockets 208 for sealing O-rings are made on both ends of the fluidic channels.

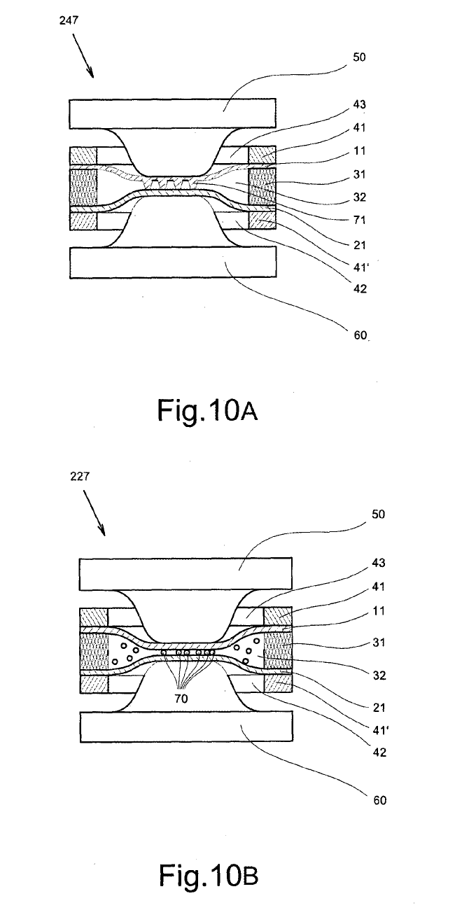

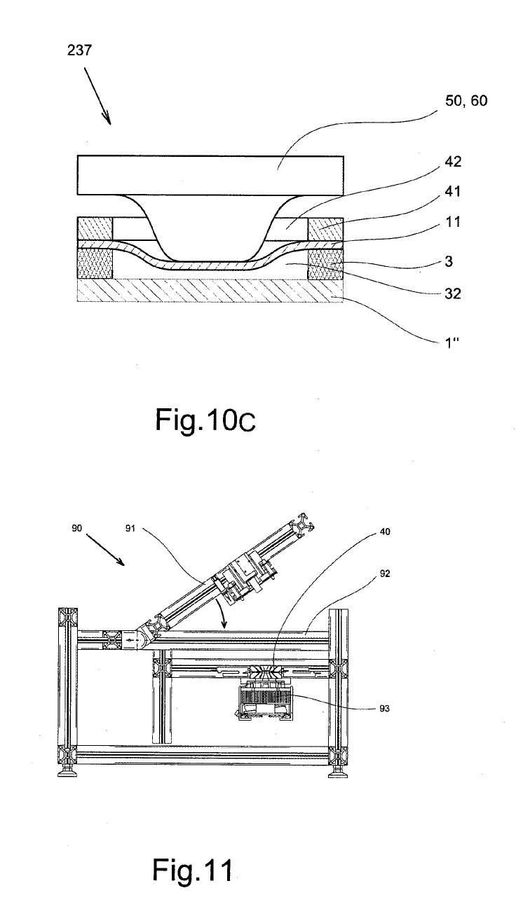

FIG. 10a shows an alternative arrangement of a sensor arrangement 247 with a film-gasket-film sandwich structure, where one of the sensor carriers 11 comprises spacers 71 on its sensor surface 204. When the sensor arrangement 247 is compressed, the minimum distance between the sensor surfaces 204 of the two films 1, 1′ is defined by the height of the spacers 71 over the base surface of the film 1. The minimum distance between the sensor surfaces 204, 204′ of the films 1, 1′ of the first and second sensor carriers 11, 21 of the sensor arrangement 247 is limited by spacers 71 made on one of the films 1, where the distance of the films 1, V can not be less than the thickness of the spacers 71. When the films 1, 1′ are compressed, the minimum distance between their sensor surfaces 201, 204′ are defined by the height of the spacers 71 over the sensor side surface of the film 1.

In FIG. 10b is shown a further alternative embodiment of the film-gasket-film sandwich sensor arrangement 227, where particles 70, e.g. beads, are dispersed in the reaction fluid. When the sandwich sensor arrangement 227 is compressed together, the minimum distance between the sensor surfaces 204, 204′ of the films 1, 1′ of the first and second sensor carriers 11, 21 of the sensor arrangement 227 is limited by the respective diameters of the dispersed particles 70 in the reaction fluids.

FIG. 10c illustrates another alternative embodiment of the film-gasket-film sandwich structured sensor arrangement 237, where the second film 1″ has a substantially higher flexural rigidity than the first film 1. Typically, the first film 1 of the arrangement 237 can be made of a 150 μm thick polyester film, and the second film 1″ is made of a more rigid and/or substantially thicker material such as a polycarbonate plate. In this example, the second film has a flexural modulus of 500 GPa, where films of even higher flexural rigidity may be used. When the sandwich style sensor arrangement 237 is compressed, only the film 1 of the first sensor carrier 11 with the lower flexural rigidity is deflected whereas the other film 1″ remains in its original state.

FIG. 11 illustrates a section view of reader instrument 90 with a cartridge 40 placed in the mechanical adaptor with a clamping mechanism formed by an upper arm 91 and a lower arm 92. Also shown is the thermocycling adaptor 93.

FIG. 12 schematically illustrates an overview scheme of the entire reader instrument 90 with a cartridge 40 and the mechanical adaptor 95 or cartridge bed, the film distance adjustment adaptor 96, the fluidic adaptor 94, the measurement adaptor 97 with two electric multiplexers 217, 218, and the instrument controller 98.

To run an analyte detection assay the cartridge 40 with a film-gasket-film sandwich structured arrangement 207 is placed into the reader instrument 90. Such reader instrument 90 comprises a housing in which the following principal components are arranged:

The reader instrument 90 comprises a mechanical adaptor 95 or cartridge bed which accommodates the cartridge 40 and enables mechanical compression of the film-gasket-film sandwich 207 between the two holder elements 41, 41′, thus sealing the volume of the chamber 32 in the arrangement 207. The mechanical adaptor 95 is typically equipped with a manual, electromagnetic or other compression mechanism, such as a lever, electromagnet or similar.

The reader instrument 90 further comprises a fluidic adaptor 94 which enables delivery and removal of the analyte fluid 210 to and from the cartridge 40, and thus to and from the chamber 32 in the sandwich arrangement 207. The fluidic adapter 94 is equipped with suitable reagent reservoir 941, fluid delivery mechanisms 942 such as pumps, connecting tubing and valves, and fluidic interfaces 943, 944 to the cartridge 40 and a second reservoir 945 for collecting the used fluidic reagents. The specific arrangement 207 of the fluidic adapter depends on the type of the assay.

The reader instrument 90 further comprises a film distance adjustment adaptor 96, including two tips 50, 60 as described in FIG. 7 and FIG. 8 with planar bases which are inserted, from both sides, into the central openings 42, 43 of the holder elements 41, 41′ of the cartridge 40, until they get into contact with the non-sensor surfaces 205, 205′ of the films 1, 1′. The film distance adjustment adaptor 96 further includes a manual and/or mechatronic actuator, typically a piezoelectrically driven translation stage, which drives one of the tips 50, 60 and allows for positioning the tip 50, 60 in the direction normal to the sandwich plane, with positioning accuracy better than 1 μm. When one tip 50, 60 moves towards the film after getting into contact with its surface, it deflects one of the films 1, 1′. The useful travel range of the tip 50, 60 is from the point of the first mechanical contact, up to the point where the sensor surface 204 of the first film 1 gets into mechanical contact with the sensor surface 204′ of the second film 1′ in the sandwich 207. Since the deformation of the film 1 is elastic and therefore reversible, the film 1 returns to its unloaded, and thus planar, state after detracting the one tip 50, 60.

Finally, the reader instrument 90 comprises a measurement adaptor 97. If impedance and/or capacitance detection is used, the measurement adaptor 97 consists of two electrical multiplexers 217, 218, the first multiplexer 217 selecting one of the electrical connections to the sensor elements 4 on the first sensor carrier 11 in the sandwich 207, and the second multiplexer 218 selecting the electrical connections to the sensor elements 4 on the second sensor carrier 21 of the arrangement 207. The combination of two selected sensor elements 4, each of those being arranged on one of the sensor carriers 11, 21, then represents a specific junction in the matrix of junctions. The measurement adaptor 97 further includes an impedance and/or capacitance measurement circuit 219, which measures impedance and/or capacitance between the two selected sensor elements 4, i.e. of the selected junction.

For alternative readout methods, an alternative embodiment of the reader instrument 90 can be equipped with alternative measurement adaptors, such as optical scanner or similar.

The reader instrument 90 has a thermocycling adaptor 93. If the assay involves PCR amplification, the thermocycling adaptor 93 provides periodic heating and cooling with defined temperature-time steps. The thermocycling adaptor 93 typically includes a thermoelectric element 61, for instance a Peltier element, which is attached with one side to an air cooler and with the other side to the tip 50. The tip 50 is inserted into the central opening 42 of one holder elements 41 of the cartridge 40, until it gets into contact with the non-sensor surface 205 of one of the sensor carriers 11, 21. When the tip 50 is heated or cooled, the thermal energy is conductively transferred to the film 1 and thus to the analyte fluid 210 in the chamber 32 in the sandwich 207. It is of advantage to build the tip 50 in a truncated conical or pyramidal form with one base being adapted to the form of the thermoelectric element 61, and with the other base being adapted to the form of the window area 6 of the film 1. This design allows for optimal conductive heat transfer and reduced energy loss. The tip 50 used for heating and cooling can, at the same time, be part of the distance adjustment adaptor 96 and fulfill both functions.

The reader instrument 90 further comprises an instrument controller 98 which is typically a microprocessor unit that controls the automated assay and readout process and communicates the measured data to downstream data processing, analysis and storage systems. The instrument controller is connected to the fluidic adaptor 94, distance adjustment adaptor 96 and the measurement adaptor 97, and can therefore control the processes of measurement.

If the analyte fluid 210 is filled in the chamber 32 through the fluidic openings 33, 34, it gets into contact with the window area 6 of both sensor carriers 11, 21, and thus to the sensor elements 4 and their functionalized surfaces 9, so a simultaneous binding or hybridization reaction between analyte molecules and probe reaction partners can occur.

In a typical application, the probe reaction partners are oligonucleotides which can specifically hybridize with DNA strands present in the analyte. Since, typically, the DNA strands are substantially longer than the oligonucleotides, the DNA strands can hybridize with more than one oligonucleotide simultaneously. For example, a DNA strand in the analyte can hybridize with one oligonucleotide on its 3′-end (this oligonucleotide will be called 3′-end or forward probe in the following), which is with its 5′-end covalently bound at one surface, and with another oligonucleotide of opposite directionality on its 5′-end (in the following, this oligonucleotide will be called 5′-end or reverse probe) which is with its 3′-end covalently bound at the other surface. Only specific DNA strands (in particular, those with their nucleotide sequences matching the 3′-end probe and 5′-end probe molecules) hybridize as a function of temperature. If the 3′-end probe and the 5′-end probe are immobilized on the surfaces of the biosensor, which are exposed to the analyte, under suitable balanced physical and chemical conditions, the complementary matching DNA strands in the analyte will first hybridize and form a bridge between the 3′-end probes and the 5′-end probes. Thus, only these DNA strands will remain bound to the biosensor surface through the 3′-end and 5′-end probes. When the rest of the analyte is removed (typically, washed away from the surface), the remaining DNA molecules can be detected and/or quantified. If more than one 3′-end probe type and more than one 5′-end probe type are immobilized on the surface, different combinations of 3′-end and 5′-end hybridizations are possible (assuming respective matching DNA strands are present in the analyte). If the specific 3′-end probe and 5′-end probe molecules are immobilized on separate sites of the biosensor, then the matching DNA strands predominantly bind at these sites only. Since the site is specific for a given hybridization type, selective hybridization detection and/or quantification is possible.

The hybridization of DNA sequences can only take place if the distance of the immobilized 3′-end probe molecule to the 5′-end probe molecule is shorter than the length of the respectively matching DNA strand. Thus, additional selection of the matching DNA strands is possible, based on specific mutual distance between the immobilized 3′-end and 5′-end probe molecules.

It is possible to immobilize the probe reaction partners, typically the 3′-end and 5′-end probes, at different surfaces of the sensor. In particular, it is possible to immobilize the primers on two planar surfaces, respectively, which are parallel to each other, and both are exposed to the analyte. If the distance of the parallel surfaces is less than the length of the matching DNA strand, then hybridization reaction between the two surfaces is possible. By controlling the distance between the parallel surfaces the upper limit of the DNA length which is able to hybridize can be specified.

For example, in a right-handed B-DNA double helix, the stacked base pairs are separated by about 3.24 angstroms (0.324 nm). Therefore, the axial length of 10·000 base pairs long b-DNA double helix is 10·000×0.324 nm=3·240 nm. If the distance between the two surfaces is about 3 μm, hybridization as described above can in principle only occur for molecules longer than 10·000 base pairs. Additional limitations may also apply.

For a multiplex study, it is of advantage to immobilize the different 3′-end and 5′-end probes in linear structures on the first and the second parallel surface, respectively. If the linear structures (stripes, lines, etc.) are arranged in a way that all rows on the first surface cross all columns on the second surface, then any junction (crossing) between any row on the first surface and any column on the second surface is a specific reaction site with specific 3′-end a 5′-end probes. Such junctions form a mesh (matrix), with rows represented by the lines of the first surface, and the columns represented by the lines of the second surface. Although, in theory, a reaction may take place between any probe immobilized along a given first-surface line, and any probe immobilized along a given second surface line, for sterical reasons only the junction with the minimum distance between the given lines is of practical importance. Thus, an assay with simultaneous hybridizations in different junctions within the said matrix is possible.

Several principles are known in the state-of-the-art to detect and/or quantify an outcome of the hybridization on one surface or between two surfaces. Labeled and unlabeled readout principles can be used for detection. In a labeled method, the single or double strand DNA is amplified by using chemical or physical labeled agent (reporter). The chemical or physical agents are then utilized for detection. Typically, luminescent molecules become chemically attached to unidentified DNA strands in the analyte solution. The assay is performed with the aim to detect in the analyte the presence of specific DNA strands, which only hybridize with specific 3′-end and 5′-end probes. The analyte is incubated in a chamber with two surfaces which contain attached probes as described above. The hybridization reaction is only enabled at the respectively matching probes, i.e. only in specific junctions of the matrix. After an incubation period, the rest of the analyte (containing DNA molecules which did not match any of the probes in the given junction matrix) is washed away. The matching DNA strands, which are already labeled with luminescent agents, remain bound to both, the first or the second surfaces at specific junctions. At suitable excitation, the luminescent labels emit observable radiation (typically, at visible light wavelengths), and the said junctions (i.e. matching probe pairs) can be identified.

It is further possible to improve the level of detection and/or enhance the quantification range by selective amplification. Typically, amplification of DNA strands can be achieved by polymerase chain reaction (PCR). In contrast to a 5′-end hybridization probe a 3′-end primer must be used which is covalently bound by its 5′-end and can be extended at its free 3′-site. 3′-primers like probes are always covalently bound by their 5′-ends.

Detection methods based on using various labels (reporter molecules) have been proven to be time-consuming, expensive and difficult to implement. However, similar detection and molecule identification is also possible without preliminary labeling the molecules in the analyte and/or other reaction components. The label-free detection and quantification methods use the intrinsic chemical or physical properties of the DNA strands themselves. Typically, DNA strands in the analyte are amplified prior to detection, as the change in chemical and/or physical properties of the DNA strands (or of the reaction environment) related to the presence of single (or a low number of) DNA molecules is too weak to be detected, so the label-free detection is typically connected with preceded or subsequent amplification.

A typical label-free detection method known in the state-of-the-art uses the local change in electrical impedance of the analyte liquid in the site where the DNA strands cumulate. This is due to substantial change in dielectric constant of a concentrated DNA (or similar organic polymer) solution as compared to a solution which does not contain DNA or similar compound. Therefore, measuring of the local electrical impedance can be used to identify the sites with concentrated DNA strands. Electrical impedance spectroscopy (EIS) is typically being used to detect the frequency-dependent impedance pattern of the junction.

More specifically, the impedance between two isolated electrodes which build one junction is of capacitance type. Therefore, the capacitance between two isolated electrodes, which are arranged in such way that the junction is filled with potentially amplified DNA strands can be measured. DNA strands can be a substantial part of the dielectric within the junction area. The capacitance measurement can be used to detect and/or quantify the DNA content. The electrodes are isolated, since the dielectric between them is typically formed by water or an electrolyte solution, which is required for hybridization and/or amplification of the DNA. Since the capacitance of a simple plate capacitor is directly proportional to the area of the plates and indirectly proportional to their distance, it is important to arrange the electrodes so that there are very close to each other, thus increasing the nominal capacitance to the range typical for standard capacitance measurement techniques in the electronics.

If the capacitance measurement technique is to be used for the readout, it is of advantage to combine the arrangement of the electrical conductors 2 to be used for capacitance measurement with the surfaces to be used for immobilizing the 3′-end probes/primers and the 5′-end probes/primers. The linear structures arranged on the first surface and embedding the 3′-end probes/primers, and linear structures arranged on the second surface and embedding the 5′-end probes/primers, can be made in such a way that they can be used as electrodes of the capacitors. More specifically, the linear structures can be made as linear, typically metallic, conductors, embedded partially or fully in the first and second surface, and covered by an insulating layer which separates the conducting metal from the volume occupied by the fluid analyte. The insulating layer can, simultaneously, serve as a layer containing chemical binding sites where the 3′-end probes/primers and the 5′-end probes/primers can be immobilized. Thus, the reaction partners are immobilized directly above the electrode. If the first and the second surfaces are approached to each other to a suitable distance, then conditions are created for the hybridization reaction to take place, and, simultaneously, a capacitor is created with a dielectric directly dependent from the amount of the reaction product.

Alternatively to the readout using electrical impedance/capacitance, a similar film can be used with other readout techniques. Optical readout can be used if the (unidentified) DNA strands in the analyte are labeled with luminescent agents as described above. The DNA strands matching any of the probe/primer combinations in the matrix junctions remain bound to both the first and the second surfaces at the said junctions. At suitable excitation, the luminescent labels emit observable radiation (typically, at visible light wavelengths). For this readout technique, a film is used with sensor elements not containing any metallic electrodes. Since the film is made from a transparent material, the luminescence originating in the junction on the sensor side of the film can be detected when observed from the non-sensor side of the film. Semiconductor lasers or multispectral lamps combined with band pass filters are used to excite the luminescence from the non-sensor side of the film, and standard luminescence detectors known in the art such as photomultipliers, CCD or CMOS cameras or similar are used to detect the luminescence on the non-sensor side of the film.

In the following, an example of an assay process is illustrated in detail with sensor carriers 11, 21, an arrangement, a cartridge 40 and a reader instrument 90 as described above. The first and the second functionalized sensor carriers 11, 21 and the gasket 31 are arranged in a sandwich structured arrangement 207, where the sensor surfaces 204, 204′ of the films 1, 1′ of the sensor carriers 11, 21 are facing each other and are at the inner surface of the chamber 32 formed between the sensor carriers 11, 21 and the gasket 31. The arrangement 207 is placed in a cartridge 40 comprising two holder elements 41, 41′.

Alternatively, the process of assembly can begin with placing a first sensor carrier 11 on a lower holder element 41 containing positioning pins 209. The film 1 of the first sensor carrier 11 is attached to the lower holder element 41, with the sensor surface 204 facing up, being aligned with the positioning pins 209. Then, the gasket 31, the second sensor carrier 21 are attached, the sensor surface 204′ of the second sensor carrier 21 facing down towards the chamber 32. The upper holder element 41′ is attached to the lower holder element 41 forming the cartridge 40. The lower holder element 41 and upper holder element 41′ are held together by magnetic forces of the permanent magnets 214.

The cartridge 40 is placed into the reader instrument 90 and is aligned in the mechanical adaptor 95 or cartridge bed. The connector pads 201 of the first film 1 and the second film 1′ of the sensor arrangement 207 are electrically connected with the measurement adaptor 97 of the reader instrument. Each of the sensor elements 4 of the first film 1 is connected to one of the inputs of the first multiplexer 217 of the measurement adaptor 97, and each of the sensor elements 4 of the second film 1′ is connected to the inputs of the second multiplexer 218 of the measurement adaptor 97. In this very example analogue multiplexers are used as first multiplexer 217 and second multiplexer 218.

The cartridge 40 is fluidically connected to the fluidic adaptor 94 of the reader instrument 90. This is typically accomplished simultaneously with sealing of the chamber 32 of the arrangement 207, and with sealing of the fluidic connection between the sensor carriers 11, 21 of the arrangement 207 and film ends of the fluidic channels 44, 45 in the holder element 41, and with sealing of the fluidic connection between the instrument ends of the fluidic channels 44, 45 in the holder element 41, and the fluidic channels on the reader instrument 90. The described sealing effects are simply achieved by exerting compression force to the holder elements 41, 41′ of the cartridge 40 in the direction normal to the plane of the films 1, 1′ of the sensor carriers 11, 21. The compression is done by manually, electromagnetically, or other similarly driven clamping mechanism.

The analyte fluid 210 is filled into the chamber 32 of the sandwich 207 using the fluidic adaptor 94. Since, in relaxed or unloaded state, the distance between the first film 1 and the second film 1′ is substantially higher than the distance necessary for chemical reaction between the two surfaces, it is substantially easier to fill the analyte fluid 210 into the chamber.

The film distance adjustment adaptor 96 is used to set up the distance between the sensor surfaces 204, 204′ of the films 1, 1′ of the first sensor carrier 11 and the second sensor carrier 21 of the sensor arrangement 207 to a value which is optimal for the hybridization or the other required reaction. The distance adjustment is enabled by the relatively low flexural modulus of the material of the film 1 of the sensor carriers 11, 21, which can be deformed easily, e.g. like a thin membrane, while clamped at the margins between the gasket 31 and holder element 41. The force required to deform and/or deflect the film 1, 1′ in the direction normal to the film surface is generated by the contact of the tip 50, 60 to the film 1, and moving of the tip 50, 60 in the direction towards the second film 1′. In this way, the films 1, 1′ of the sensor carriers 11, 21 approach each other, and the volume of the chamber 32 decreases, i.e. a negative deformation direction. Alternatively, the deflection can also be forced by pressure difference between the analyte fluid 210 on the sensor surface 204 of the film 1, 1′, and the air or any other medium on the non-sensor surface 205 of the film 1, 1′. In this way, deflection in both positive and negative direction, i.e. increasing and decreasing the volume of the chamber 32, respectively, is possible.

Several effects can be achieved by the elastic deflection of the films 1, 1′: If the films 1, 1′ get closer to each other, the diffusion path length for the molecules from the fluid phase of the volume in the chamber 32 to the direction normal to one of the sensor surface 204, 204′ gets shorter, which accelerates the reaction. To enable specific reaction type, such as a simultaneous binding of a molecule to the sensor compounds 5 immobilized at both surfaces 204, 204′ of the films 1, 1′, the distance should not exceed specific value so approaching of the films 1, 1′ is of principal importance. If impedance and/or capacitance measurement is used a readout method, the distance between the film 1, 1′ is affecting the baseline impedance and/or capacitance of the capacitor formed between the electrical conductors 2, 2′. If this distance is increased, the electric fields between the conductors 2, 2′ might get shielded through formation of electric double layers formed by ions in solution to such extent that effective measurement of differences caused by local deposition of detected substances becomes unfeasible.

If PCR amplification is required, the thermocycling adaptor 93 is activated, and the analyte fluid 210, which contains also all components required for PCR like polymerase, dNTP and further additives, in the chamber 32 of the sandwich 207 is periodically heated and cooled according to a specific time/temperature pattern, which leads to amplification of the specifically hybridized molecules.

Additional fluidic processes, such as exchanging the reaction fluid after partial PCR amplification, washing, etc., can be applied during the assay if specified. In particular, the reagents contained in the analyte which did not participate at the reaction and have not been bound by the immobilized primers, can be removed from the chamber 32.

After PCR amplification, the amplicons are concentrated and bound in the junctions of the junction matrix with matching 5′-end and 3′-end primers. Different matching amplicons can be concentrated in a single junction, while some junctions may remain empty. The concentration of the amplicons in different junctions is, primarily, dependent on the original concentration of the potentially matching reaction partners in the analyte.

The measurement adaptor 97 is activated. The first multiplexer 217 connects the first sensor element 4 of the first film 1 to the first input of the impedance and/or capacitance measurement circuit 219. The second multiplexer 218 connects the first sensor element 4 of the second film 1′ to the second input of the impedance and/or capacitance measurement circuit 219. Impedance and/or capacitance is measured in this configuration, giving the data for the ‘first sensor element-first sensor element’ junction. This measurement readout is stored. The first multiplexer 217 then connects the second sensor element 4 of the first film 1 to the first input of the impedance and/or capacitance measurement circuit 219, and the impedance and/or capacitance is measured and stored for the ‘second sensor element-first sensor element’ junction. The process is repeated for all sensor elements 4 of the first film 1 and all sensor elements 4 of the second film 1′, so impedance and/or capacitance readouts are stored for all junctions of the junction matrix.

The readouts are analyzed with respect to the known allocation of 3′-end and 5′-end probes/primers on the sensor elements 4 of the first film 1 and the second film 1′, and detection and/or quantification results are produced.

In the following, an alternative second example of the invention is provided by template specific PCR amplification in an assembly of two ultra high density flexible circuit boards with functionalized electrical conductors 2, 2′. Unless otherwise stated, this alternative example is identical with the first example.

Structured PET films which contain 24 electrical conductors 2 each were designed as described in the first example of the invention. The film 1 was heat treated polyethylene terephthalate (PET) substrate. The electrical conductors 2 were made of nickel carrying a top layer of gold, and measured 25 μm in width and approximately 11 mm in length inside the window area 6. The electrical conductors 2 are leading from the window area 6 to the connector pads 201 of the electrical connector 3. The surfaces of the PET support and electrical conductors 2, 2′ formed by metal of the electrodes were on level which means that the surface of the structured PET films is entirely flat.

The electrical conductors 2, 2′ are electrically insulated and functionalized with different sensor compounds 5, namely oligonucleotides functioning as PCR primers, through a combination of electrode guided electrodeposition and polymerization. The sensor compounds 5, in this example primer molecules are used, are bound to the polymer via spacer molecules and therefore surface exposed and accessible.

For the coating process a cartridge 40 of 50×50×20.5 mm outer dimension analogue to the description in the first example has been assembled containing one 24 electrode PET film 1, 1′, one 250 μm thick gasket 31 and one stainless steel counter electrode. A zero-insertion force electrical connector 3 and flat cable was used to electrically connect the film 1, 1′ to a switch board through which one or several electrodes could be set to a predetermined potential. The chamber 32 in the window area 6 confined a volume of approximately 25 μl. The solutions for the functionalization were successively channeled through the chamber 32. During an exposure time the functionalization of specific electrical conductors 2, 2′ was carried out. Between each functionalization the chamber 32 has been rinsed thoroughly with water. After the functionalization the cartridge has been disassembled to remove the functionalized film 1, 1′ before rinsing it thoroughly with water, drying it with a stream of nitrogen and placing it into an oven for curing. The coating was inspected by fluorescence scanning and exemplarily by AFM scanning. Electrodes were homogenously coated with functionalizing films of approximately 1 μm thickness.

Then, a sensor arrangement 207 comprising two of such individually functionalized sensor carriers 1, 1′ was assembled with a 250 μm thick gasket in a cartridge 40 as described in the first example. The cartridge 40 was transferred into, and aligned by the mechanical adaptor of the reader instrument 90. The connector pads 201 of the electrical connectors 3 were electrically connected with the measurement adaptor 97 of the reader instrument 90.

The fluidic channels 44, 45 connect the outer side of the cartridge 40 with the chamber in the arrangement 207. Only the fluidic channels 45 of the upper holder 41′ are used, the connection between the film and the lower holder 41 is blinded by replacing a sealing O-ring with a matching size silicone disc. Then, the outer side of the cartridge 40 was connected to the fluidic adaptor 94 of the reader instrument. Through controlled pumping of solutions either a continuous stream of liquid or aliquots in the order of 25 μl at the time could be flushed through or injected into the chamber 32. The aqueous solutions contained buffer compounds, template DNA or a PCR mix with TaqHS polymerase, dNTP and additives.

The electrical connectors 3 of the first and the second film 1, 1′ of the sensor arrangement 207 are connected via zero-insertion force connectors. A relaxed state, where the two flexible functionalized films had no physical contact, and a loaded state, where the two flexible functionalized films were in physical contact, could be imposed alternatively through a piezo-driven height manipulation device (distance adjustment adaptor 96) which was brought and stayed in contact with the upper film. The lower film was in contact with a Peltier element connected metal block, the thermocycling adaptor 93.

The relaxed state was used to facilitate fast solution exchange during washing and filling of the measurement cell. The separation between the two films 1, 1′ in the centre and around the edges was approximately 250 μm similar to the height of the gasket. Under this condition solutions could pass the cell with flow rates of approximately 1 ml/min which accounts for approximately one nominal exchange of the volume in 1.5 sec. Tests with colored solutions have shown that solution exchanges proceed rather evenly across the entire surface in the open state, slightly faster along the central axis between the inlet and outlet port, slightly slower along the edges, slowest at the corners. In the loaded state solution exchange would proceed significantly slower and essentially only along the edges where the first and seconds films 1, 1′ diverge to the distance which is determined by the height of the gasket, here 250 μm. The difference of the solution exchange rate between the middle and the edge of the window area 6 would be very large. Therefore, in the unloaded state the sensor arrangement 207 was first washed with ultrapure water before it was filled with 25 μl of a solution containing 1.5 ng/μl DNA template sequence of 350 bp length, 0.08 u TaqHS polymerase, and 0.1 mM dNTP, 0.2 μg/ml BSA, 3 mM Mg²⁺, 0.3 M Trehalose in a Taq Hotstar buffer (Cryogene, US). The template contained complementary sequences to the primers F and R. The primers N were not complementary to partial sequences within the template.

Then, the loaded state was applied through the piezo driven positioning system, the film distance adjustment adaptor 96, which moved the first film 1 to reach close contact with the second film 1′. Since the electrical conductors 2, 2′ are embedded in the supporting PET polymer, and are surmounted just by the functionalizing coating layer, the approach of the surfaces stopped when the curved coatings touch each other. Each electrical conductor 2 of the first film contacts each electrical conductor 2′ of the second film in a very small point shaped area, thereby forming a single sensor. Small deviations from the perfect even flat surface alignment are mediated by a thin gel layer which is situated between the first film 1 and the tip 50. The gel layer levels small height variations and ensures that each electrode junction is compressed with a moderate pressure which is small enough to not indent the functionalized coating completely.

The reference impedances of all combinations of electrical conductors 2, 2′ from the first and second film 1, 1′ were measured successively with a scanning routine which set all non-participating electrical conductors 2, 2′ on shielding ground.

The outcome of the measurement is typically presented in form of an impedance matrix diagram. The rows R0 to R23 of the impedance matrix represent the electrical conductors 2 of the first film 1, whereas the columns C0 to C23 of the impedance matrix represent the electrical conductors 2′ of the second film 1′. The matrix cells stand for the individual junctions. Impedance is measured for all individual sensors of the sensor arrangement at a given frequency. In this example, a frequency of 1 MHz was used. The modulus component of each of the complex impedance values is determined.

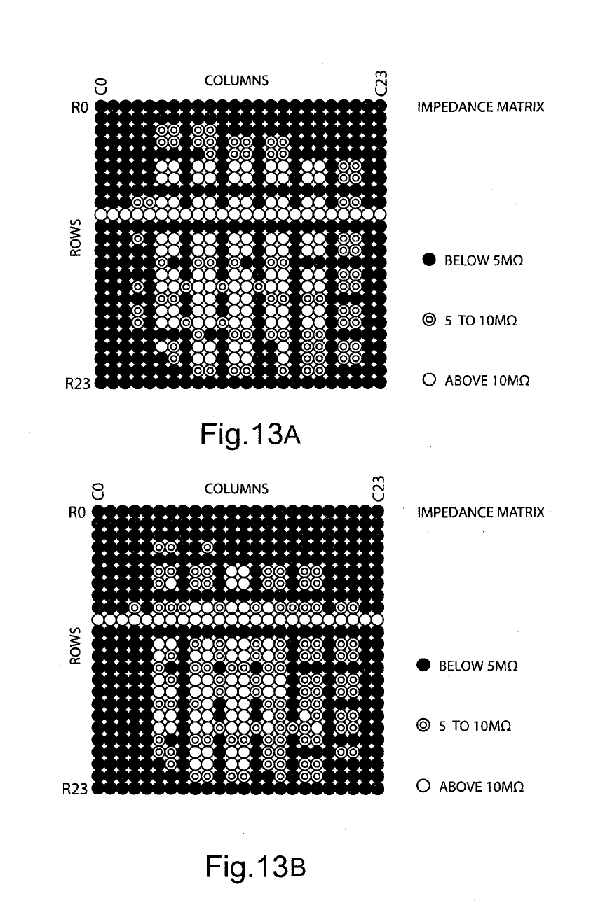

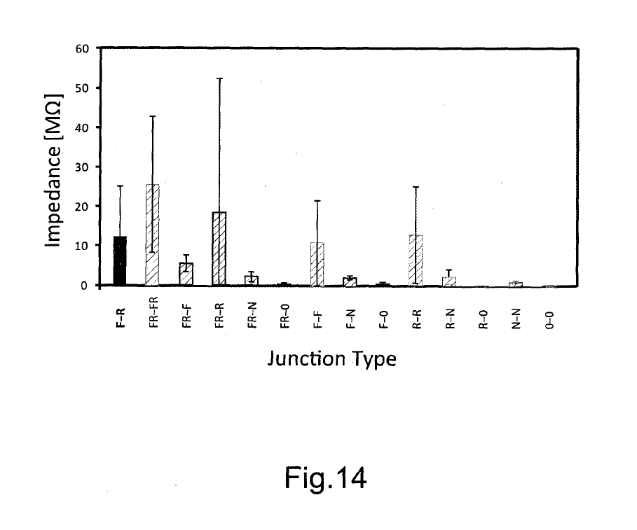

In FIGS. 13a and 13b, such diagrams are shown in low impedance resolution, discriminating three impedance modulus ranges only. In this example, threshold values of 5 MOhm, i.e. mega Ohms, and 10 MOhm are used. Sensors with an impedance value higher than 10 MOhm are depicted by white circles, sensors with an impedance modulus between 5 MOhm and 10 MOhm are depicted by concentrical circles and sensors with an impedance modulus lower than 5 MOhm are depicted by black circles. Higher impedance, i.e. lower capacitance at given measurement frequency, means higher concentration of the hybridized and amplified substances in the given sensor junction. Note that the impedance is actually measured with a substantially higher resolution, and the diagrams have been simplified for publication purposes.

The regular pattern of the impedance which is seen in FIG. 13a is caused by the different functionalization of the electrodes. The significant differences are seen between sensors elements 4 at junctions of electrodes which carry the primers, F, R or N, and junctions of electrical conductors 2, 2′ which have no primers in their respective coatings 9. Further, also in the loaded state 40 PCR cycles were conducted by thermocycling the sensor arrangement 207 alternating between a denaturing step for 45 sec at 95° C., an annealing step for 30 sec at 57° C. and an extension step for 60 sec at 72° C. After the PCR cycling the second impedance scan was recorded comprising again all combinations of electrical conductors 2, 2′. Results are shown in FIG. 13b. The impedimetric scans before and after the PCR display a change in signal intensity as function of the ability of the primers to hybridize and amplify the particular template.

As explained above, in a typical application, the targeted molecules in the analyte hybridize to the forward primers F which are immobilized in the first film 1 with their respective 5′-site. During the first extension the primers F are extended in 5′- to 3′-direction and produce complementary copies of the analyte molecules which are now covalently bound to the first film 1 via the primer binding site. After the subsequent melting step and during the following annealing step the complementary copies are capable to hybridize to the reverse primers R which are immobilized in the second film 1′ with the opposite end. For a given molecule species, a junction containing matching forward primers in the first film 1 and matching reverse primers in the second film 1′, is denoted as “F-R”, wherein the letter(s) before and after dash specifying substances in the first and second film 1, 1′, respectively. For experimental purposes, it is of advantage to check the general ability of the matching molecules to hybridize to the primers specifically and selectively, i.e., to hybridize to the matching primers irrespectively of their physical location, and not to bind to non-matching primers or to junctions with no primers. Therefore, in our experiments, we also used junctions which are functionalized as follows:
‘FR-FR’, i.e. both the first film and the second film contain a mixture of forward and reverse primers,
‘FR-F’ and ‘FR-R’ wherein the first film is functionalized with a primer mixture comprising forward and reverse primers, the second film only contains forward or reverse primer, respectively,
‘FR-N’, wherein the first film is functionalized with a forward and reverse primer mixture, the second film only contains a non-matching primer,
‘F-F’ (‘R-R’) wherein the first film is functionalized with a forward (reverse) primer only, and the second film is functionalized with a forward (reverse) primer only,
‘F-N’, forward primer in the first film, non-matching primer in the second film,
‘R-N’, reverse primer in the first film, non-matching primer in the second film,
‘N-N’, non matching primers in both films and
‘0-0’ zero indicating that no primers are deposited on the respective coatings,
as well as all combinations of any of the primers with no primers.

The combinatorial approach of the matrix structure implies that all possible primer combinations can be realized in symmetrical matrices. The statistical evaluation of all equivalent junctions from the pool of in total 576 junctions is shown as mean values with the respected confidence interval in FIG. 14. The impedance modulus represents the change of the impedance in relation to the change of the dielectric inside the electrode junctions as a function of the present sensor or primer molecules. The largest changes are seen at junctions with combinations of F- and R-primers. The smallest change is seen at junctions without primers. Junctions which contain primers N which are not complement to the sequence of the template display also very little change. The results demonstrate the label free detection of DNA by impedimetric scanning of “nanogap junctions”.

The highest concentration of the hybridized molecules at a given junction is expected for the ‘FR-FR’ configuration, since multiple hybridization combinations (first film-first film, second film-second film, first film-second film) are possible. FIG. 14 shows that this assumption is fulfilled. It is also shown that the first film-second film hybridization, which is the desired functionality is preferred over single surface hybridization, the ‘F-R’ junctions having higher mean impedance than the ‘FR-F’ junctions. The ‘FR-R’ junctions have higher mean impedance than the ‘F-R’, however a substantially higher deviation. The mean impedance modulus of any of the non-matching combinations, i.e. ‘F-N’, ‘R-N’, ‘N-N’, and ‘0-0’, is significantly below the value for any of the matching configurations.

The disclosed examples of biosensors may be used for multiplex assays in molecular biology, in particular, for assays requiring selective detection and/or quantification of analyte constituents which react specifically with two probes simultaneously. The probes are part of the biosensor and are specific for a given biosensor type. If more than two probes are included in the biosensor, then the biosensor can be used for multiplexed studies, in that more than one combination of the probes can react with different analyte constituents, so more than one analyte constituent can be specifically detected and/or quantified simultaneously.

### REFERENCES CITED

### Journal Publications

Adessi C, Matton G, Ayala G, Turcatti G, Mermod J J, Mayer P, Kawashima E. 2000 Solid phase DNA amplification: characterisation of primer attachment and amplification mechanisms. Nucleic Acids Res. 28e87
Focke M, Kosse D, Müller C, Reinecke H, Zengerle R, von Stetten F., 2010 Lab-on-a-Foil: microfluidics on thin and flexible films. Lab Chip. 10(11):1365-86
Jia G, Siegrist J, Deng C, Zoval J V, Stewart G, Peytavi R, Huletsky A, Bergeron M G, Madou M J. 2007 A low-cost, disposable card for rapid polymerase chain reaction. Colloids Surf B Biointerfaces. 58(1):52-60
Khan Z, Poetter K, Park D J., 2008 Enhanced solid phase PCR: mechanisms to increase priming by solid support primers. Anal Biochem. 375(2):391-3
Liu Y, Rauch C B, Stevens R L, Lenigk R, Yang J, Rhine D B, Grodzinski P. 2002 DNA amplification and hybridization assays in integrated plastic monolithic devices. Anal Chem. 74(13):3063-70
Saiki R, Scharf S, Faloona F, Mullis K, Horn G, Erlich H, Arnheim N. 1985 Enzymatic amplification of beta-globin genomic sequences and restriction site analysis for diagnosis of sickle cell anemia. Science 230 (4732): 1350-1354

### Patent Publications

WO 2010104479, Electrical sensor for ultrasensitive nucleic acid detection
PCT/AT2012/000043, Junction array sensor device
U.S. Pat. No. 6,878,345, Ultra high throughput bioassay screening system
U.S. Pat. No. 5,863,502A1, Parallel reaction cassette and associated devices

1-26. (canceled)

27. A sensor carrier, comprising:
a flexible film having surfaces and a window area; and
a plurality of functionalized sensor elements each having a functional layer and supported by said flexible film, said functional layers disposed on a same one of said surfaces of said flexible film within said window area, wherein a region of each of said functional layers of said functionalized sensor elements is functionalized with at least one sensor compound.

28. The sensor carrier according to claim 27, wherein:
said flexible film is formed of a film material having a flexural modulus from 0.5 GPa to 2 GPa; and/or
said flexible film has a thickness of between 10 um to 300 um; and/or
said flexible film is composed of a polymer selected from the group consisting of polyethylene, polyethylene terephtalate, polyimide, polyethylene naphthalate, polycarbonate, polyether sulphone, polyarylate and mixtures thereof.

29. The sensor carrier according to claim 28, wherein a flexibility and elasticity of said film material of said flexible film, and/or a flexibility and elasticity of a material of said functional layers, and/or a size and/or a shape of said window area, are chosen so that, when a boundary of said window area is fixed in a direction normal to a plane of said flexible film, and said window area is deflected and/or deformed, out of the plane of said flexible film in the direction normal to the plane of said flexible film, at least one part of said surface is pushed by 10 to 500 um, into the direction normal to the plane of said flexible film, said flexible film is deflected elastically.

30. The sensor carrier according to claim 27, wherein at least said window area of said flexible film is transparent.

31. The sensor carrier according to claim 27, further comprising spacers disposed on a surface of the sensor carrier on a side of said functional layers, said spacers are elevated on the surface of the sensor carrier by 0.05 um to 1 um.

32. The sensor carrier according to claim 27, wherein said sensor compound contains oligonucleotides binding to binding sites of analyte molecules.

33. The sensor carrier according to claim 27, further comprising at least one of:
a plurality of electrical conductors, each of said electrical conductors is assigned to one of said sensor elements, and each of said electrical conductors is disposed underneath said functional layer of said sensor element in or on said flexible film; or
an electrical connector disposed on said flexible film, said electrical connector having a plurality of connector pads, each of said connector pads being connected to one of said electrical conductors and/or said electrical conductors in or on said flexible film contain a material selected from the group consisting of nickel, copper, gold, carbon, indium tin oxide, and semiconductors.

34. The sensor carrier according to claim 33, wherein said flexible film has a rectangular shape, said electrical conductors are disposed in parallel in at least said window area of said flexible film, and at least one portion of each of said sensor elements and/or each of said electrical conductors is inclined to edges of said flexible film by an angle of 40° to 50°.

35. A sensor configuration, comprising:
a sensor carrier having a first flexible film with surfaces and a first window area and a plurality of functionalized first sensor elements each having a first functional layer and supported by said first flexible film, said first functional layers disposed on a same one of said surfaces of said first flexible film within said first window area, wherein a first region of each of said first functional layers of said functionalized first sensor elements is functionalized with at least one first sensor compound;
a gasket having an inner edge and a central opening formed therein;
a second film having a plurality of functionalized second sensor elements each being formed by a second functional layer and a second window area, said second functional layers disposed on a same surface of said second film within said second window area, wherein a second region of each of said second functional layers of said second sensor elements is functionalized; and
at least one second sensor compound disposed in a respective one of said second regions of said second sensor elements, said second film having a plurality of line shaped electrical conductors and one electrical connector and/or spacers disposed on a surface, wherein said gasket is disposed between said sensor carrier and said second film, so that a chamber is formed between said sensor carrier and said second film, and said chamber is surrounded by said inner edge of said gasket.

36. The sensor configuration according to claim 35, wherein said second film has a material with a flexural modulus higher than 5 GPa, and/or said at least said second window area of said second film is transparent.

37. A sensor configuration, comprising:
two sensor carriers each having a flexible film with surfaces and a window area and a plurality of functionalized sensor elements each having a functional layer and supported by said flexible film, said functional layers disposed on a same one of said surfaces of said flexible film within said window area, wherein a region of each of said functional layers of said functionalized sensor elements is functionalized with at least one sensor compound, said two sensor carriers further having conductors; and
a gasket having an inner edge and a central opening formed therein, said gasket disposed between said two sensor carriers, so that a chamber is formed between said two sensor carriers and surrounded by said inner edge of said gasket, and said surfaces of said two sensor carriers, on which said sensor elements are disposed, are facing each other and each of said sensor elements and/or said conductor of one of said two sensor carriers opposes or faces at least one said sensor element and/or said conductor of an opposite of said two sensor carriers.

38. The sensor configuration according to claim 37, further comprising one of particles or beads disposed between said surfaces being sensitive opposing surfaces of said two sensor carriers within said chamber, wherein said particles or said beads are included or dispersed in an analyte fluid containing analyte molecules to be analyzed, wherein a minimum distance between said surfaces of said two flexible films is defined by a diameter of said particles or said beads.

39. The sensor configuration according to claim 37, wherein each of said two sensor carriers and said gasket has an inlet and an outlet, said inlet and said outlet leading through at least one of said two sensor carriers and/or in said gasket, leading from an outside to said chamber and enabling fluid to stream into said chamber or out of said chamber through said inlet and said outlet.

40. A cartridge, comprising:
a sensor configuration according to claim 37;
holder elements, said gasket and said sensor carriers or one of said sensor carriers and one said flexible film are fixed and compressed by said holder elements so that said chamber is confined by said gasket, and/or said two sensor carriers and/or said flexible film, so that said chamber is sealed and impermeable to liquid.

41. The cartridge according to claim 40, wherein each of said holder elements has at least one opening formed therein, wherein said opening is covered by one part of one of said two sensor carriers, said part at least partially confining said chamber.

42. The cartridge according to claim 40, wherein each of said holder elements has two opposing openings formed therein and including a first opening and a second opening, said first opening is covered by one portion of one of said two sensor carriers, and said second opening is covered by one portion of the other of said two sensor carriers or said flexible film, said portions of said sensor carriers or said flexible film at least partially confining said chamber.

43. The cartridge according to claim 40, wherein:
at least one of said two sensor carriers has an inlet and an outlet formed therein; and
each of said holder elements further has at least two channels including a first channel and a second channel, said first channel connects said inlet with an outer surface of said holder element and said second channel connects said outlet with an outer surface of said holder element.

44. The cartridge according to claim 40, wherein:
said sensor carriers and said gasket or said sensor carrier and one of said flexible films and said gasket are fixed between said holder elements; and
said holder elements have protrusions and holes formed therein matching each other when said holder elements are assembled; and
said sensor carriers, said flexible film and/or said gasket have positioning holes formed therein and being penetrated by at least some of said protrusions.

45. The cartridge according to claim 40, wherein said holder elements compressing said sensor configuration to seal said chamber.

46. A method for measuring a concentration of an analyte compound in an analyte fluid, which comprises the steps of:
providing an elastically flexible first film and an elastically flexible second film with a first sensor compound being disposed on a region of the first film and a second sensor compound being disposed on a region of the second film, the first and second sensor compounds being disposed on opposing sensor surfaces of the first and second films facing each other, wherein the first sensor compound provided for binding or adhering to a first portion of a molecule of the analyte compound and the second sensor compound provided for binding or adhering to a second portion of the molecule of the analyte compound;
disposing the analyte fluid in a chamber formed between and surrounded by the first and second films, wherein the first film and the second film are elastically deflected or deformed in a manner, that a distance between the opposing sensor surfaces of the first and second films facing each other is reduced and equals or is smaller than a distance between the first portion and the second portion of the molecule of the analyte compound so that a sensor is formed between the region of the first film on which the first sensor compound is disposed and the region of the second film on which the second sensor compound is disposed, where said regions of the first and second films are disposed to face each other; and
measuring an amount of molecules bound or adhered to the first and second sensor compounds.

47. The method according to claim 46, which further comprises adding one of particles or beads of a given diameter to the analyte fluid, wherein the particles or the beads are filled into the chamber between the first and second films so that a minimum distance between the sensor surfaces of the first and second films is defined by the diameter of the particles or the beads.

48. The method according to claim 46, which further comprises measuring a capacitance or an impedance between electrical conductors in the regions of the first film and the second film on which the first and second sensor compounds are disposed, and the capacitance or the impedance indicating an amount of the analyte compound in the analyte fluid.

49. The method according to claim 46, wherein before setting the distance of the opposing first and second films the distance of the first and second films is increased in order to increase an amount of the analyte fluid within the chamber and/or the distance of the first and second films is regulated to adjust an internal volume of the chamber in order to control an amount of the analyte fluid accommodated in the chamber.

50. The method according to claim 46, which further comprises repeatedly changing the distance between the first and second films before or during a measurement so that the analyte fluid in the chamber is mechanically agitated, so that by adjusting a flow in the chamber a specific shear force distribution profile is achieved in the chamber.

51. The method according to claim 46, which further comprises changing the distance between the first and second films in order to pump fluid into the chamber or out of the chamber.

52. A method for measuring a concentration of an analyte compound in an analyte fluid, which comprises the steps of:
providing a measurer selected from the group consisting of two sensor carriers according to claim 27, a sensor configuration according to claim 35 and a cartridge according to claim 40;
forming a plurality of sensors between the regions of the first film on which the first sensor compound or a plurality thereof is disposed and the regions of the second film on which the second sensor compound or plurality thereof is disposed, where the regions are disposed to face each other; and
determining a concentration of analyte compounds located or deposited between two facing regions of a sensor separately for each sensor.
